SEQUENCE LISTING (8 sequences)

SEQ ID NO: 1 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1208 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 3 - protein, 192 aa, SARS-CoV-2
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAP

SEQ ID NO: 4 - protein, 1208 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 5 - protein, 1255 aa, Human coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 6 - protein, 222 aa, Human coronavirus
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNF

SEQ ID NO: 7 - protein, 1353 aa, Human coronavirus
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFQKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 8 - protein, 120 aa, Human coronavirus
CDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINK

(19) United States
(12) Patent Application Publication
(10) Pub. No.: US 2022/0305112 A1
(43) Pub. Date: Sep. 29, 2022

(54) NOVEL METHODS AND USES

(71) Applicant: GLAXOSMITHKLINE BIOLOGICALS SA, Rixensart (BE)

(72) Inventor: Robbert Gerrit VAN DER MOST, Rixensart (BE)

(73) Assignee: GLAXOSMITHKLINE BIOLOGICALS SA, Rixensart (BE)

(21) Appl. No.: 17/680,433

(22) Filed: Feb. 25, 2022

(30) Foreign Application Priority Data
Mar. 29, 2021 (EP) 21165695.4

(51) Int. Cl. A61K 39/215; A61K 39/39

(52) U.S. Cl. CPC A61K 39/215; A61K 39/39; A61K 2039/55566

(57) ABSTRACT

The present invention relates to immunisation with a coronavirus spike antigen and a squalene emulsion adjuvant to elicit broad immune responses, and to related aspects.

### TECHNICAL FIELD

The present invention relates to immunisation with a coronavirus spike antigen and a squalene emulsion adjuvant to elicit broad immune responses, and to related aspects.

### BACKGROUND ART

Coronaviruses are spherical and enveloped, positive-sense single-stranded RNA viruses. They have the largest genomes (26-32 kb) among known RNA viruses, and are phylogenetically divided into four genera (alpha, beta, gamma, delta), with betacoronaviruses further subdivided into four lineages (A, B, C, D). Coronaviruses infect a wide range of avian and mammalian species, including humans. Of the seven known coronaviruses to emerge in the human population, four of them (HCoV-OC43 (betacoronavirus), HCoV-229E (alphacoronavirus), HCoV-HKU1 (betacoronavirus) and HCoV-NL63 (alphacoronavirus)) are known to circulate annually in humans and generally cause mild upper respiratory diseases in immunocompetent hosts, although severe infections can be caused in infants, young children, elderly individuals, and the immunocompromised. Both HCoV-OC43 and HCoV-HKU1 cause self-limiting, common cold-like illnesses. (Wang, 2020) In contrast, the Middle East respiratory syndrome coronavirus (MERS-CoV) and the severe acute respiratory syndrome coronavirus 1 (SARS-CoV-1), belonging to betacoronavirus lineages C and B, respectively, are highly pathogenic (Cui, 2019).

It is unclear whether the latest betacoronavirus to emerge in the human population, severe acute respiratory syndrome coronavirus 2 (SARS-CoV-2), also of lineage B, will circulate annually in humans. SARS-CoV-2, like MERS-CoV and SARS-CoV-1, is highly pathogenic. MERS-CoV, SARS-CoV-1, and SARS-CoV-2 all crossed the species barrier into humans and caused outbreaks of severe, often fatal, respiratory diseases. (Letko, 2020)

Coronavirus disease 2019 (COVID-19) is an infectious disease caused by SARS-CoV-2. The disease was first identified in late 2019 and has spread globally. The World Health Organization (WHO) declared the 2019-2020 coronavirus outbreak a Public Health Emergency of International Concern (PHEIC) on 30 Jan. 2020 and a pandemic on 11 Mar. 2020. The time from exposure to onset of symptoms is typically around five days but may range from two to fourteen days. While the majority of cases result in mild symptoms, some progress to viral pneumonia and multi-organ failure. As of 17 Mar. 2021, more than 120 million cases have been reported, resulting in more than 2.66 million deaths (WHO, 17 Mar. 2021).

Preliminary data suggest that antibody responses to the spike (S) protein particularly the receptor binding domain (RBD) of SARS-CoV-2 correlate with protection against disease and viral load.

Candidate vaccines under clinical development include a subunit vaccine comprising the SARS-CoV-2 spike protein receptor binding domain (RBD) displayed on a two-component protein nanoparticle, known as RBD-NP (Walls, 2020). RBD-NP has been combined with a squalene emulsion (Essai O/W 1849101); a tocopherol-containing squalene emulsion (AS03); a TLR-7 agonist adsorbed to aluminium hydroxide (AS37); a TLR-9 agonist formulated with aluminium hydroxide (CpG 1018-Alum); or aluminium hydroxide alone. All five adjuvants induced substantial neutralizing antibodies (nAb) and CD4 T cell responses in non-human primates after two administrations. AS03, CpG 1018-Alum, AS37 and aluminium hydroxide groups conferred significant protection to the non-human primates against SARS-CoV-2 infection, with nAb titers highly correlated with protection against infection. (Arunachalam, 2021)

A prefusion stabilised spike trimer having a transmembrane deletion (preS dTM) formulated with tocopherol-containing squalene emulsion and administered twice to non-human primates provided significant protection in the upper and lower airways from high dose SARS-CoV-2 challenge (Francica, 2021)

VIR-7831 and VIR-7832 antibodies have been shown to neutralise wild-type SARS-CoV-2 in vitro as well as pseudo-viruses encoding variant spike proteins from B.1.1.7, B.1.351 and P.1 variants. The VIR-7831/VIR-7832 epitope does not overlap with mutational sites in the current variants of concern and continues to be highly conserved among circulating sequences. (Cathcart, 2021)

Oil-in-water emulsion adjuvants containing squalene have featured in licensed pandemic and prepandemic influenza vaccines. ‘AS03’ (WO2006/100109; Garcon, 2012; Cohet, 2019) includes squalene, alpha-tocopherol and polysorbate 80. An adult human dose of AS03_{A} contains 10.69 mg squalene, 11.86 mg alpha-tocopherol and 4.86 mg polysorbate 80 (Fox, 2009; Morel, 2011). Certain reduced does of AS03 have also been described (WO2008/043774), including AS03_{B} (½ dose), AS03_{C} (¼ dose) and AS03_{D} (⅛ dose) (Carmona Martinez, 2014). AS03 and MF59 (a submicron oil-in-water emulsion of squalene, polysorbate 80 and sorbitan trioleate) adjuvants have been shown to augment the immune responses to 2 doses of an inactivated H7N9 influenza vaccine, with the tocopherol containing AS03-adjuvanted formulations inducing the highest titers (Jackson, 2015). Adjuvantation with AS03 leads to a number of differences in the B cell receptor repertoire induced by influenza vaccination (Galson, 2016). Furthermore, priming with AS03 adjuvanted H5N1 influenza vaccine improved the kinetics, magnitude and durability of the immune response after a heterologous booster vaccination (Leroux-Roels, 2010) and the induction of CD4 T cell responses during AS03 adjuvanted influenza vaccination was found to be important in preparing the immune system for antigens of diverse strains (van der Most, 2014).

Stable emulsions (SE) have also been described which contain squalene, phospholipid, poloxamer 188 (Pluronic F68) and glycerol in ammonium phosphate buffer (Carter, 2016). The SE have sometimes been described as containing low levels of alpha-tocopherol as an antioxidant (Sun, 2017).

Viral evolution is generating mutations in the spike protein which could compromise the effectiveness of vaccines (Mahase, 2021; Wang, 2021). Consequently, there remains a need for the provision of immunisation approaches which can mitigate the impact of mutations in the spike protein on vaccine protection.

### SUMMARY OF THE INVENTION

Squalene emulsion adjuvants are of benefit in conjunction with a coronavirus spike antigen.

The invention therefore provides a method for the prophylaxis of infection by a first coronavirus in a human subject, the method comprising administering to the subject (i) a coronavirus spike antigen derived from a second coronavirus, and (ii) a squalene emulsion adjuvant. Further provided is a method for inducing a cross-reactive immune response against a first coronavirus in a human subject, the method comprising administering to the subject (i) a coronavirus spike antigen derived from a second coronavirus, and (ii) a squalene emulsion adjuvant.

The invention also provides a squalene emulsion adjuvant for use in the prophylaxis of infection by a first coronavirus in a human subject by administration with a coronavirus spike antigen derived from a second coronavirus. Also provided is a squalene emulsion adjuvant for use in eliciting a cross-reactive immune response against a first coronavirus in a human subject by administration with a coronavirus spike antigen derived from a second coronavirus.

The invention also provides a coronavirus spike antigen derived from a second coronavirus for use in the prophylaxis of infection by a first coronavirus in a human subject by administration with a squalene emulsion adjuvant. Also provided is a coronavirus spike antigen derived from a second coronavirus, for use in eliciting a cross-reactive immune response against a first coronavirus in a human subject by administration with a squalene emulsion adjuvant

The invention also provides the use of a squalene emulsion adjuvant in the manufacture of a medicament for use in the prophylaxis of infection by a first coronavirus in a human subject by administration with a coronavirus spike antigen derived from a second coronavirus. Also provided is the use of a squalene emulsion adjuvant in the manufacture of a medicament for use in eliciting a cross-reactive immune response against a first coronavirus in a human subject by administration with a coronavirus spike antigen derived from a second coronavirus.

The invention also provides the use of a coronavirus spike antigen derived from a second coronavirus in the manufacture of a medicament for use in the prophylaxis of infection by a first coronavirus in a human subject by administration with a squalene emulsion adjuvant. Also provided is the use of a coronavirus spike antigen derived from a second coronavirus in the manufacture of a medicament for use in eliciting a cross-reactive immune response against a first coronavirus in a human subject by administration with a squalene emulsion adjuvant.

The invention also provides an immunogenic composition comprising: (i) coronavirus spike antigen derived from a second coronavirus, and (ii) a squalene emulsion adjuvant, for use in the prophylaxis of infection by a first coronavirus in a human subject. Additionally provided is an immunogenic composition comprising: (i) coronavirus spike antigen derived from a second coronavirus, and (ii) a squalene emulsion adjuvant, for use in inducing a cross-reactive immune response against a first coronavirus in a human subject. Also provided is a kit comprising: (i) a first container comprising a coronavirus spike antigen derived from a second coronavirus; and (ii) a second container comprising a squalene emulsion adjuvant. Additionally provided is a kit comprising: (i) a first container comprising a coronavirus spike antigen derived from a second coronavirus; (ii) a second container comprising a squalene emulsion adjuvant, (iii) instructions for combining the coronavirus spike antigen (such as a single dose of the coronavirus spike antigen) with the squalene emulsion adjuvant (such as a single dose of the squalene emulsion adjuvant) to produce an immunogenic composition prior to administration of a single dose of the immunogenic composition to a subject.

The invention also provides the use of (i) a coronavirus spike antigen derived from a second coronavirus, and (ii) a squalene emulsion adjuvant, in the manufacture of a medicament for use in the prophylaxis of infection by a first coronavirus in a human subject. Further provided is the use of (i) a coronavirus spike antigen derived from a second coronavirus, and (ii) a squalene emulsion adjuvant, in the manufacture of a medicament for use in inducing a cross-reactive immune response against a first coronavirus in a human subject.

### BRIEF DESCRIPTION OF THE SEQUENCES

SEQ ID NO: 1: SARS-CoV-2 S protein

SEQ ID NO: 2: SARS-CoV-2 S protein ectodomain

SEQ ID NO: 3: SARS-CoV-2 S protein receptor binding domain

SEQ ID NO: 4: Pre-fusion stabilised SARS-CoV-2 S protein ectodomain

SEQ ID NO: 5: SARS-CoV-1 S protein UniProtKB Accession No. P59594-1 dated 23 Apr. 2003

SEQ ID NO: 6: SARS-CoV-1 S protein receptor binding domain

SEQ ID NO: 7: MERS-CoV Spike glycoprotein GenBank Accession No. AFS88936.1 Version 1 dated 4 Dec. 2012

SEQ ID NO: 8: MERS-CoV Spike glycoprotein receptor binding domain

### BRIEF DESCRIPTION OF THE FIGURES

FIG. 1: Schematic of the SARS-CoV-2 Spike (S) protein primary structure by domain (from Wrapp, 2020). SS, signal sequence; NTD, N-terminal domain; RBD, receptor binding domain; SD1 , subdomain 1; SD2, subdomain 2, S1/S2, S1/S2 protease cleavage site; S2′, S2′ protease cleavage site; FP, fusion peptide; HR1, heptad repeat 1; CH, central helix; CD, connector domain; HR2, heptad repeat 2; TM, transmembrane domain; CT, cytoplasmic tail. Arrows denote protease cleavage sites.

FIG. 2: Schematic of selected SARS-CoV-2 lineages indicating 36 of 880 lineages containing 68% of 560,000 samples tested by Public Health England.

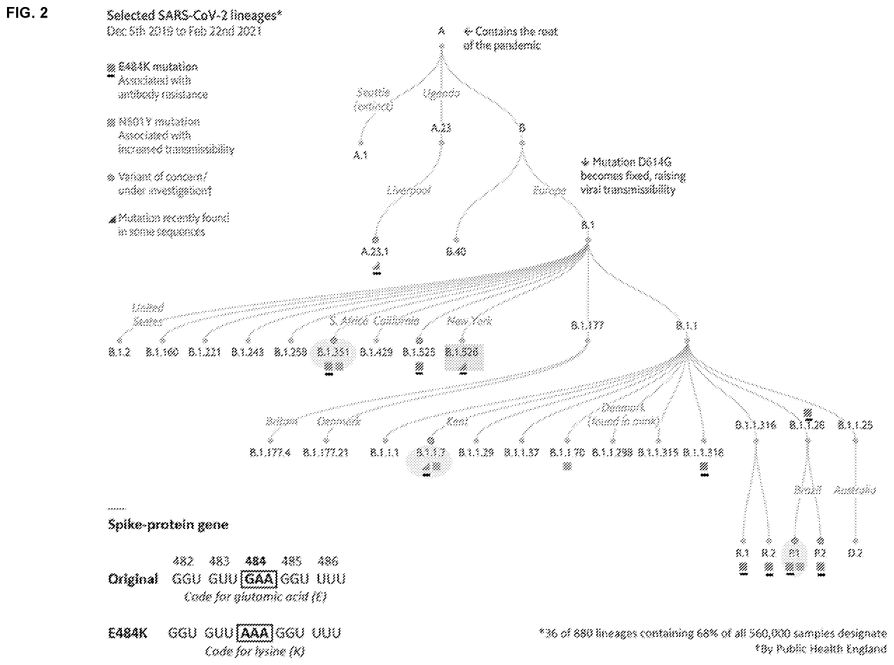

### DETAILED DESCRIPTION OF THE INVENTION

### Squalene Emulsion Adjuvants

The term ‘squalene emulsion adjuvant’ as used herein refers to a squalene-containing oil-in-water emulsion adjuvant. The term ‘tocopherol-containing squalene emulsion adjuvant’ as used herein refers to a squalene- and tocopherol-containing oil-in-water emulsion adjuvant wherein the weight ratio of squalene to tocopherol is 20 or less (i.e. 20 weight units of squalene or less per weight unit of tocopherol or, alternatively phrased, at least 1 weight unit of tocopherol per 20 weight units of squalene). Tocopherol-containing squalene emulsion adjuvants are therefore a subset of squalene emulsion adjuvants and are of particular interest in the present invention.

Squalene is a branched, unsaturated terpenoid ([CH₃)₂C[═CHCH₂CH₂C(CH₃)]₂═CHCH₂—]₂; C₃₀H₅₀; 2,6,10,15,19,23-hexamethyl-2,6,10,14,18,22-tetracosahexaene; CAS Registry Number 7683-64-9). Squalene is readily available from commercial sources or may be obtained by methods known in the art. Squalene shows good biocompatibility and is readily metabolised.

Squalene emulsion adjuvants will typically have a submicron droplet size. Droplet sizes below 200 nm are beneficial in that they can facilitate sterilisation by filtration. There is evidence that droplet sizes in the 80 to 200 nm range are of particular interest for potency, manufacturing consistency and stability reasons. (Klucker, 2012; Shah, 2014; Shah, 2015; Shah, 2019). Suitably the squalene emulsion adjuvant has an average droplet size of less than 1 um, especially less than 500 nm and in particular less than 200 nm. Suitably the squalene emulsion adjuvant has an average droplet size of at least 50 nm, especially at least 80 nm, in particular at least 100 nm, such as at least 120 nm. The squalene emulsion adjuvant may have an average droplet size of 50 to 200 nm, such as 80 to 200 nm, especially 120 to 180 nm, in particular 140 to 180 nm, such as about 160 nm.

Uniformity of droplet sizes is desirable. A polydispersity index (PdI) of greater than 0.7 indicates that the sample has a very broad size distribution and a reported value of 0 means that size variation is absent, although values smaller than 0.05 are rarely seen. Suitably the squalene emulsion adjuvant has a polydispersity of 0.5 or less, especially 0.3 or less, such as 0.2 or less.

The droplet size, as used herein, means the average diameter of oil droplets in an emulsion and can be determined in various ways e.g. using the techniques of dynamic light scattering and/or single-particle optical sensing, using an apparatus such as the Accusizer™ and Nicomp™ series of instruments available from Particle Sizing Systems (Santa Barbara, USA), the Zetasizer™ instruments from Malvern Instruments (UK), or the Particle Size Distribution Analyzer instruments from Horiba (Kyoto, Japan). See Light Scattering from Polymer Solutions and Nanoparticle Dispersions Schartl, 2007. Dynamic light scattering (DLS) is the preferred method by which droplet size is determined. The preferred method for defining the average droplet diameter is a Z-average i.e. the intensity-weighted mean hydrodynamic size of the ensemble collection of droplets measured by DLS. The Z-average is derived from cumulants analysis of the measured correlation curve, wherein a single particle size (droplet diameter) is assumed and a single exponential fit is applied to the autocorrelation function. Thus, references herein to average droplet size should be taken as an intensity-weighted average, and ideally the Z-average. PdI values are easily provided by the same instrumentation which measures average diameter.

In order to maintain a stable submicron emulsion, one or more emulsifying agents (i.e. surfactants) are generally required. Surfactants can be classified by their ‘HLB’ (Griffin's hydrophile/lipophile balance), where a HLB in the range 1-10 generally means that the surfactant is more soluble in oil than in water, whereas a HLB in the range 10-20 means that the surfactant is more soluble in water than in oil. HLB values are readily available for many surfactants of interest or can be determined experimentally, e.g. polysorbate 80 has a HLB of 15.0 and TPGS has a HLB of 13 to 13.2. Sorbitan trioleate has a HLB of 1.8. When two or more surfactants are blended, the resulting HLB of the blend is typically calculated by the weighted average e.g. a 70/30 wt % mixture of polysorbate 80 and TPGS has a HLB of (15.0×0.70)+(13×0.30) i.e. 14.4. A 70/30 wt % mixture of polysorbate 80 and sorbitan trioleate has a HLB of (15.0×0.70)+(1.8×0.30) i.e. 11.04.

Surfactant(s) will typically be metabolisable (biodegradable) and biocompatible, being suitable for use as a pharmaceutical. The surfactant can include ionic (cationic, anionic or zwitterionic) and/or non-ionic surfactants. The use of only non-ionic surfactants is often desirable, for example due to their pH independence. The invention can thus use surfactants including, but not limited to:
the polyoxyethylene sorbitan ester surfactants (commonly referred to as the Tweens or polysorbates), such as polysorbate 20 and polysorbate 80, especially polysorbate 80;
copolymers of ethylene oxide (EO), propylene oxide (PO), and/or butylene oxide (BO), sold under the DOWFAX™, Pluronic™ (e.g. F68, F127 or L121 grades) or Synperonic™ tradenames, such as linear EO/PO block copolymers, for example poloxamer 407, poloxamer 401 and poloxamer 188;
octoxynols, which can vary in the number of repeating ethoxy (oxy-1,2-ethanediyl) groups, with octoxynol-9 (Triton X-100, or t-octylphenoxypolyethoxyethanol) being of particular interest;
(octylphenoxy)polyethoxyethanol (IGEPAL CA-630/NP-40);
phospholipids such as phosphatidylcholine (lecithin);
polyoxyethylene fatty ethers derived from lauryl, cetyl, stearyl and oleyl alcohols (known as Brij surfactants), such as polyoxyethylene 4 lauryl ether (Brij 30, Emulgen 104P), polyoxyethylene-9-lauryl ether and polyoxyethylene 12 cetyl/stearyl ether (Eumulgin™B1, cetereth-12 or polyoxyethylene cetostearyl ether);
sorbitan esters (commonly known as the Spans), such as sorbitan trioleate (Span 85), sorbitan monooleate (Span 80) and sorbitan monolaurate (Span 20);
or tocopherol derivative surfactants, such as alpha-tocopherol-polyethylene glycol succinate (TPGS).

Many examples of pharmaceutically acceptable surfactants are known in the art e.g. see Handbook of Pharmaceutical Excipients 6th edition, 2009. Methods for selecting an optimising the choice of surfactant used in a squalene emulsion adjuvant are illustrated in Klucker, 2012. In general, the surfactant component has a HLB between 10 and 18, such as between 12 and 17, in particular 13 to 16. This can be typically achieved using a single surfactant or, in some embodiments, using a mixture of surfactants. Surfactants of particular interest include: poloxamer 401, poloxamer 188, polysorbate 80, sorbitan trioleate, sorbitan monooleate and polyoxyethylene 12 cetyl/stearyl ether either alone, in combination with each other or in combination with other surfactants. Especially of interest are polysorbate 80, sorbitan trioleate, sorbitan monooleate and polyoxyethylene 12 cetyl/stearyl ether either alone, or in combination with each other. A particular surfactant of interest is polysorbate 80. A particular combination of surfactants of interest is polysorbate 80 and sorbitan trioleate. A further combination of surfactants of interest is sorbitan monooleate and polyoxyethylene cetostearyl ether.

In certain embodiments the squalene emulsion adjuvant comprises one surfactant, such as polysorbate 80. In some embodiments the squalene emulsion adjuvant comprises two surfactants, such as polysorbate 80 and sorbitan trioleate or sorbitan monooleate and polyoxyethylene cetostearyl ether. In other embodiments the squalene emulsion adjuvant comprises three or more surfactants, such as three surfactants.

The amount of squalene in a single dose, such as a human dose, of squalene emulsion adjuvant may be 50 mg or less, especially 40 mg or less, in particular 30 mg or less, such as 20 mg or less (for example 15 mg or less). The amount of squalene in a single dose, such as a human dose, of squalene emulsion adjuvant may be 0.5 mg or more, especially 1 mg or more, in particular 2 mg or more, such as 4 mg or more and desirably 8 mg or more. The amount of squalene in a single dose, such as a human dose, of squalene emulsion adjuvant may be 0.5 to 50 mg, especially 1 to 20 mg, in particular 2 to 15 mg, such as 5 to 15 mg. The amount of squalene in a single dose, such as a human dose, of squalene emulsion adjuvant may be 0.5 to 2 mg, 2 to 4 mg, 4 to 8 mg, 8 to 12 mg, 12 to 16 mg, 16 to 20 mg or 20 to 50 mg.

The amount of squalene in a single dose, such as a human dose, of squalene emulsion adjuvant may be 1.2 to 20 mg, in particular 1.2 to 15 mg. The amount of squalene in a single dose, such as a human dose, of squalene emulsion adjuvant may be 1.2 to 2 mg, 2 to 4 mg, 4 to 8 mg or 8 to 12.1 mg. For example, the amount of squalene in a single dose, such as a human dose, of squalene emulsion adjuvant may be 1.21 to 1.52 mg, 2.43 to 3.03 mg, 4.87 to 6.05 mg or 9.75 to 12.1 mg.

Typically the weight ratio of squalene to surfactant is 0.73 to 6.6, especially 1 to 5, in particular 1.5 to 4.5. The weight ratio of squalene to surfactant may be 1.5 to 3, especially 1.71 to 2.8, such as 2.2 or 2.4. The weight ratio of squalene to surfactant may be 2.5 to 3.5, especially 3 or 3.1. The weight ratio of squalene to surfactant may be 3 to 4.5, especially 4 or 4.3.

The amount of surfactant in a single dose, such as a human dose, of squalene emulsion adjuvant is typically at least 0.4 mg. Generally, the amount of surfactant in a single dose, such as a human dose, of squalene emulsion adjuvant is 18 mg or less. The amount of surfactant in a single dose, such as a human dose, of squalene emulsion adjuvant may be 0.4 to 9.5 mg, in particular 0.4 to 7 mg. The amount of surfactant in a single dose, such as a human dose, of squalene emulsion adjuvant may be 0.4 to 1 mg, 1 to 2 mg, 2 to 4 mg or 4 to 7 mg. For example, the amount of surfactant in a single dose, such as a human dose, of squalene emulsion adjuvant may be 0.54 to 0.71 mg, 1.08 to 1.42 mg, 2.16 to 2.84 mg or 4.32 to 5.68 mg.

The squalene emulsion adjuvant may contain one or more tocopherols. Any of the α, β, γ, δ, ε and/or ξ tocopherols can be used, but α-tocopherol (also referred to herein as alpha-tocopherol) is typically used. D-alpha-tocopherol and D/L-alpha-tocopherol can both be used. Tocopherols are readily available from commercial sources or may be obtained by methods known in the art. In some embodiments the squalene emulsion adjuvant does not contain tocopherol. In some embodiments the squalene emulsion adjuvant contains tocopherol (i.e. at least one tocopherol, suitably one tocopherol), especially alpha-tocopherol, in particular D/L-alpha-tocopherol.

Tocopherols have been used, in relatively small amounts, in squalene emulsion adjuvants as antioxidants. Desirably tocopherols are present a level where the weight ratio of squalene to tocopherol is 20 or less, such as 10 or less. Suitably the weight ratio of squalene to tocopherol is 0.1 or more. Typically the weight ratio of squalene to tocopherol is 0.1 to 10, especially 0.2 to 5, in particular 0.3 to 3, such as 0.4 to 2. Suitably, the weight ratio of squalene to tocopherol is 0.72 to 1.136, especially 0.8 to 1, in particular 0.85 to 0.95, such as 0.9. Alternatively, the weight ratio of squalene to tocopherol is 3.4 to 4.6, especially 3.6 to 4.4, in particular 3.8 to 4.2, such as 4.

The amount of tocopherol in a single dose, such as a human dose, of squalene emulsion adjuvant is typically at least 0.5 mg, especially at least 1.3 mg. Generally, the amount of tocopherol in a single dose, such as a human dose, of squalene emulsion adjuvant is 55 mg or less. The amount of tocopherol in a single dose, such as a human dose, of squalene emulsion adjuvant may be 1.3 to 22 mg, in particular 1.3 to 16.6 mg. The amount of tocopherol in a single dose, such as a human dose, of squalene emulsion adjuvant may be 1.3 to 2 mg, 2 to 4 mg, 4 to 8 mg or 8 to 13.6 mg. For example, the amount of tocopherol in a single dose, such as a human dose, of squalene emulsion adjuvant may be 1.33 to 1.69 mg, 2.66 to 3.39 mg, 5.32 to 6.77 mg or 10.65 to 13.53 mg.

In certain embodiments the squalene emulsion adjuvant may consist essentially of squalene, tocopherol (if present), surfactant and water. In addition to squalene, tocopherol, surfactant and water, squalene emulsion adjuvants may contain additional components as desired or required depending upon the intended final presentation and vaccination strategy, such as buffers and/or tonicity modifying agents, for example modified phosphate buffered saline (disodium phosphate, potassium biphosphate, sodium chloride and potassium chloride).

A squalene emulsion of interest in the present invention is known as ‘MF59’ (WO90/14837; Podda, 2003; Podda, 2001) and is a submicron oil-in-water emulsion of squalene, polysorbate 80 (also known as Tween 80™), and sorbitan trioleate (also known as Span 85™). It may also include citrate ions e.g. 10 mM sodium citrate buffer. The composition of the emulsion by volume can be about 5% squalene, about 0.5% polysorbate 80 and about 0.5% sorbitan trioleate. The adjuvant and its production are described in more detail in Vaccine Design: The Subunit and Adjuvant Approach (chapter 10), Vaccine Adjuvants: Preparation Methods and Research Protocols (chapter 12) and New Generation Vaccines (chapter 19). As described in O'Hagan, 2007, MF59 is manufactured on a commercial scale by dispersing sorbitan trioleate in the squalene, dispersing polysorbate 80 in an aqueous phase (e.g. citrate buffer), then mixing these two phases to form a coarse emulsion which is then microfluidised. The emulsion is typically prepared at double-strength (4.3% v/v squalene, 0.5% v/v polysorbate 80 and 0.5% v/v sorbitan trioleate) and is diluted 1:1 (by volume) with an antigen composition to provide a final adjuvanted vaccine composition. An adult human dose of MF59 contains 9.75 mg squalene, 1.17 mg polysorbate 80 and 1.17 mg sorbitan trioleate (O'Hagan, 2013). An adult human dose of MF59C.1, as used in the seasonal influenza vaccine Fluad™, contains 9.75 mg squalene, 1.175 mg polysorbate 80 and 1.175 mg sorbitan trioleate. 0.66 mg sodium citrate, 0.04 mg citric acid (O'Hagan, 2013) in 0.5 ml of water for injection (Fluad™ Summary of Product Characteristics).

A further squalene emulsion of interest in the present invention is known as ‘AF03’ (US2007/0014805; Klucker, 2012). AF03 includes squalene, sorbitan monooleate, polyoxyethylene cetostearyl ether and mannitol. AF03 is prepared by cooling a pre-heated water-in-oil emulsion until it crosses its emulsion phase inversion temperature, at which point it thermoreversibly converts into an oil-in-water emulsion. The mannitol, cetostearyl ether and a phosphate buffer are mixed in one container to form an aqueous phase, while the sorbitan ester and squalene are mixed in another container to form an oily component. The aqueous phase is added to the oily component and the mixture is then heated to approximately 60° C. and cooled to provide the final emulsion. The emulsion is typically initially prepared as a concentrate with a composition of 32.5% squalene, 4.8% sorbitan monooleate, 6.2% polyoxyethylene cetostearyl ether and 6% mannitol and 50.5% phosphate buffered saline. AF03 adjuvant contains 12.4 mg squalene, 1.9 mg sorbitan monooleate, 2.4 mg polyoxyethylene cetostearyl ether and 2.3 mg mannitol per 500 ul human adult dose (Humenza™ Summary of Product Characteristics).

Another squalene emulsion of interest in the present invention is known as ‘AS03’ (Garçon, 2012) and is prepared by mixing an oil mixture (consisting of squalene and alpha-tocopherol) with an aqueous phase (polysorbate 80 and buffer), followed by microfluidisation (WO2006/100109). AS03 is typically prepared at double-strength with the expectation of dilution by an aqueous antigen containing composition prior to administration. An adult human dose of AS03_{A} contains 10.69 mg squalene, 11.86 mg alpha-tocopherol and 4.86 mg polysorbate 80 (Morel, 2011; Fox, 2009). Certain reduced does of AS03 have also been described (WO2008/043774), including AS03_{B} (½ dose), AS03_{C} (¼ dose) and AS03_{D} (⅛ dose) (Carmona Martinez, 2014).

As discussed above, high pressure homogenization (HPH or microfluidisation) and a phase inversion temperature method (PIT) may be applied to yield squalene emulsion adjuvants which demonstrate uniformly small droplet sizes and long-term stability. More recently, squalene based self-emulsifying adjuvant systems (SEAS) have been described. WO2015/140138 and WO2016/135154 describe the preparation of oil/surfactant compositions, which when diluted with an aqueous phase spontaneously form oil-in-water emulsions having small droplet particle sizes, such emulsions can be used as immunological adjuvants. An adult human dose of ‘SEA160’ emulsion may include 7.62 mg squalene, 2.01 mg polysorbate 80 and 2.01 mg sorbitan trioleate. (Shah, 2014; Shah, 2015; Shah, 2019)

International patent application WO2020/160080 and Lodaya, 2019 describe further squalene based self-emulsifying adjuvant systems (SEAS), specifically systems comprising a tocopherol in addition to squalene. ‘SEAS44’ contains 60% v/v squalene, 15% v/v alpha-tocopherol and 25% v/v polysorbate 80. The squalene/tocopherol/polysorbate mixture is intended to be diluted approximately 10-fold with an aqueous medium to form the final emulsion adjuvant. Consequently, an adult human dose of SEAS44 emulsion may include about 13 mg squalene, 3.6 mg alpha-tocopherol and 6.7 mg polysorbate 80.

Other squalene emulsion adjuvants have been described including:
SWE (Younis, 2018) comprising squalene 3.9% w/v, sorbitan trioleate 0.47% w/v, and polysorbate 80 (0.47% w/v) dispersed in 10 mM citrate buffer at pH 6.5. Consequently, an adult human dose of SWE may include about 9.75 mg squalene, 1.175 mg sorbitan trioleate and 1.175 mg polysorbate 80, similar to MF59.
SE (Carter, 2016; Sun, 2017) comprising squalene, phosphatidyl choline, poloxamer 188 and an ammonium phosphate buffered aqueous phase also containing glycerol. Sometimes SE has been described as containing small amounts of tocopherol. An adult human dose of SE may include about 8.6 mg squalene, 2.73 mg phosphatidyl choline and 0.125 mg poloxamer 188, optionally with 0.05 mg tocopherol.
CoVaccine (Hilgers, 2006; Hamid, 2011; Younis, 2019) comprises squalene, polysorbate 80 and sucrose fatty acid sulfate esters, typically with phosphate buffered saline. An adult human dose of CoVaccine may include about 40 mg squalene, 10 mg polysorbate 80 and 10 mg sucrose fatty acid sulfate esters.

The squalene emulsion adjuvant may be derived from MF59. Consequently, the squalene emulsion adjuvant may comprise squalene, polysorbate 80, sorbitan trioleate and water. The squalene emulsion adjuvant may consist essentially of squalene, polysorbate 80, sorbitan trioleate and water. Optionally the aqueous phase may contain additional components as desired or required depending upon the intended final presentation and vaccination strategy, such as buffers and/or tonicity modifying agents, in particular citrate ions e.g. 10 mM sodium citrate buffer.

Typically, the weight ratio of squalene to polysorbate 80 is 10 to 6.6, especially 9.1 to 7.5, in particular 8.7 to 7.9, such as 8.3.

Typically, the weight ratio of squalene to sorbitan trioleate is 10 to 6.6, especially 9.1 to 7.5, in particular 8.7 to 7.9, such as 8.3.

A single dose, such as a typical full human dose, of squalene emulsion adjuvant derived from MF59 may comprise 9 to 11 mg of squalene, such as 9.5 to 10 mg, in particular 9.75 mg. Higher or lower doses of squalene emulsion adjuvant derived from MF59 may be used. Suitably a single dose is at least 0.1× a typical full human dose, especially at least 0.25× a typical full human dose, in particular at least 0.5× a typical full human dose. Desirably the single dose is less than or equal to a full human dose. For example, the single dose may be 0.1 to 1× a typical full human dose, i.e. comprising 0.9 to 11 mg of squalene.

Particular single doses of interest include 0.1× a typical full human dose i.e. comprising 0.9 to 1.1 mg of squalene, 0.125× a typical full human dose i.e. comprising 1.1 to 1.4 mg of squalene, 0.25× a typical full human dose i.e. comprising 2.2 to 2.8 mg of squalene, such as 0.5× a typical full human dose i.e. comprising 4.5 to 5.5 mg of squalene or 1× a typical full human dose i.e. comprising 9 to 11 mg of squalene.

Squalene emulsion adjuvant derived from MF59 may include citrate ions e.g. 10 mM sodium citrate buffer.

The squalene emulsion adjuvant may be derived from AF03. Consequently, the squalene emulsion adjuvant may comprise squalene, sorbitan monooleate, polyoxyethylene cetostearyl ether and water. The squalene emulsion adjuvant may consist essentially of squalene, sorbitan monooleate, polyoxyethylene cetostearyl ether and water. Mannitol has been shown to reduce the phase transition temperature and is therefore desirable for manufacturing reasons, although excessive levels of mannitol may cause heterogeneity in size and larger droplets (Klucker, 2012). Optionally the aqueous phase may contain additional components as desired or required depending upon the intended final presentation and vaccination strategy, such as buffers and/or tonicity modifying agents, in particular phosphate buffered saline.

Typically, the weight ratio of squalene to sorbitan monooleate is 7.8 to 5.2, especially 7.15 to 5.85, in particular 6.8 to 6.2, such as 6.5.

Typically, the weight ratio of squalene to polyoxyethylene cetostearyl ether is 6.2 to 4.1, especially 5.7 to 4.7, in particular 5.4 to 4.9, such as 5.2.

Typically, the weight ratio of squalene to mannitol is 6.5 to 4.3, especially 5.9 to 4.9, in particular 5.7 to 5.1, such as 5.4.

A single dose, such as a typical full human dose, of squalene emulsion adjuvant derived from AF03 may comprise 11.2 to 13.6 mg of squalene, such as 12 to 12.8 mg, in particular 12.4 mg. Higher or lower doses of squalene emulsion adjuvant derived from AF03 may be used. Suitably a single dose is at least 0.1× a typical full human dose, especially at least 0.25× a typical full human dose, in particular at least 0.5× a typical full human dose. Desirably the single dose is less than or equal to a full human dose. For example, the single dose may be 0.1 to 1× a typical full human dose, i.e. comprising 1.1 to 13.6 mg of squalene.

Particular single doses of interest include 0.1× a typical full human dose i.e. comprising 1.1 to 1.35 mg of squalene, 0.125× a typical full human dose i.e. comprising 1.4 to 1.7 mg of squalene, 0.25× a typical full human dose i.e. comprising 2.8 to 3.4 mg of squalene, such as 0.5× a typical full human dose i.e. comprising 5.6 to 6.8 mg of squalene or 1× a typical full human dose i.e. comprising 11.2 to 13.6 mg of squalene.

Squalene emulsion adjuvant derived from AF03 may also include in particular phosphate buffered saline.

The squalene emulsion adjuvant may be derived from AS03. Consequently, the squalene emulsion adjuvant may comprise squalene, tocopherol, polysorbate 80 and water. The squalene emulsion adjuvant may consist essentially of squalene, tocopherol, polysorbate 80 and water. Optionally the aqueous phase may contain additional components as desired or required depending upon the intended final presentation and vaccination strategy, such as buffers and/or tonicity modifying agents. Suitable buffers include Na₂HPO₄ and KH₂PO₄. Suitable tonicity modifying agents include NaCl and KCl. Modified phosphate buffered saline may be used, such as comprising Na₂HPO₄ and KH₂PO₄, NaCl and KCl.

Any of the α, β, γ, δ, ε or ξ tocopherols can be used, but α-tocopherol (also referred to herein as alpha-tocopherol) is typically used. D-alpha-tocopherol and D/L-alpha-tocopherol can both be used. A particularly desirable alpha-tocopherol is D/L-alpha-tocopherol.

Typically, the weight ratio of squalene to tocopherol is 0.5 to 1.5, especially 0.6 to 1.35, in particular 0.7 to 1.1, such as 0.85 to 0.95 e.g. 0.9. Suitably the tocopherol is alpha-tocopherol, such as D/L-alpha-tocopherol.

Typically, the weight ratio of squalene to polysorbate 80 is 1.2 to 3.6, especially 1.46 to 3.3, in particular 1.9 to 2.5 such as 2.1 to 2.3 e.g. 2.2.

A single dose, such as a typical full human dose, of squalene emulsion adjuvant derived from AS03 may comprise 9.7 to 12.1 mg of squalene, such as 10.1 to 11.8 mg, in particular 10.7 mg. Higher or lower doses of squalene emulsion adjuvant derived from AS03 may be used. Suitably a single dose is at least 0.1× a typical full human dose, especially at least 0.25× a typical full human dose, in particular at least 0.5× a typical full human dose. Desirably the single dose is less than or equal to a full human dose. For example, the single dose may be 0.1 to 1× a typical full human dose, i.e. comprising 0.9 to 12.1 mg of squalene.

Particular single doses of interest include 0.1× a typical full human dose i.e. comprising 0.9 to 1.3 mg of squalene (typically with 1 to 1.4 mg tocopherol, such as D/L-alpha tocopherol, and 0.43 to 0.57 mg polysorbate 80), 0.125× a typical full human dose i.e. comprising 1.2 to 1.6 mg of squalene (typically with 1.3 to 1.7 mg tocopherol, such as D/L-alpha tocopherol, and 0.54 to 0.71 mg polysorbate 80), 0.25× a typical full human dose i.e. comprising 2.4 to 3 mg of squalene (typically with 2.6 to 3.4 mg tocopherol, such as D/L-alpha tocopherol, and 1 to 1.5 mg polysorbate 80), such as 0.5× a typical full human dose i.e. comprising 4.8 to 6.1 mg of squalene (typically with 5.3 to 6.8 mg tocopherol, such as D/L-alpha tocopherol, and 2.1 to 2.9 mg polysorbate 80) or 1× a typical full human dose i.e. comprising 9.7 to 12.1 mg of squalene (typically with 10.6 to 13.6 mg tocopherol, such as D/L-alpha tocopherol, and 4.3 to 5.7 mg polysorbate 80).

Squalene emulsion adjuvant derived from AS03 may also include in particular a phosphate buffered saline, such as modified phosphate buffered saline.

The squalene emulsion adjuvant may be derived from SE. Consequently, the squalene emulsion adjuvant may comprise squalene, phosphatidyl choline, poloxamer 188 and water, optionally with glycerol. The squalene emulsion adjuvant may consist essentially of squalene, phosphatidyl choline, poloxamer 188 and water, optionally with glycerol. Optionally the aqueous phase may contain additional components as desired or required depending upon the intended final presentation and vaccination strategy, such as buffers and/or tonicity modifying agents, in particular ammonium phosphate buffer. Tocopherol, such as alpha-tocopherol may be present as an antioxidant.

Typically, the weight ratio of squalene to phosphatidyl choline is 2.52 to 3.8, especially 2.85 to 3.5, in particular 3 to 3.3, such as 3.15.

Typically, the weight ratio of squalene to poloxamer 188 is 55 to 83, especially 62 to 76, in particular 65.5 to 72.5, such as 69.

Typically, the weight ratio of squalene to tocopherol, if present, is at least 50, especially 137 to 207, in particular 154 to 190, such as 163 to 181, for example 172.

A single dose, such as a typical full human dose, of squalene emulsion adjuvant derived from SE may comprise 7.7 to 9.5 mg of squalene, such as 8.1 to 9 mg, in particular 8.6 mg. Higher or lower doses of squalene emulsion adjuvant derived from SE may be used. Suitably a single dose is at least 0.1× a typical full human dose, especially at least 0.25× a typical full human dose, in particular at least 0.5× a typical full human dose. Desirably the single dose is less than or equal to a full human dose. For example, the single dose may be 0.1 to 1× a typical full human dose, i.e. comprising 0.77 to 9.5 mg of squalene.

Particular single doses of interest include 0.1× a typical full human dose i.e. comprising 0.77 to 0.95 mg of squalene, 0.125× a typical full human dose i.e. comprising 0.96 to 1.2 mg of squalene, 0.25× a typical full human dose i.e. comprising 1.9 to 2.4 mg of squalene, such as 0.5× a typical full human dose i.e. comprising 3.8 to 4.8 mg of squalene or 1× a typical full human dose i.e. comprising 7.7 to 9.5 mg of squalene.

Squalene emulsion adjuvant derived from SE may also include in particular ammonium phosphate buffer and glycerol.

The squalene emulsion adjuvant may be derived from SEA160. Consequently, the squalene emulsion adjuvant may comprise squalene, polysorbate 80, sorbitan trioleate and water. The squalene emulsion adjuvant may consist essentially of squalene, polysorbate 80, sorbitan trioleate and water. Optionally the aqueous phase may contain additional components as desired or required depending upon the intended final presentation and vaccination strategy, such as buffers and/or tonicity modifying agents.

Typically, the weight ratio of squalene to polysorbate 80 is 4.6 to 3.0, especially 4.2 to 3.4, in particular 4.0 to 3.6, such as 3.8.

Typically, the weight ratio of squalene to sorbitan trioleate is 4.6 to 3.0, especially 4.2 to 3.4, in particular 4.0 to 3.6, such as 3.8.

A single dose, such as a typical full human dose, of squalene emulsion adjuvant derived from SEA160 may comprise 6.8 to 8.4 mg of squalene, such as 7.2 to 8 mg, in particular 7.6 mg. Higher or lower doses of squalene emulsion adjuvant derived from SEA160 may be used.

Suitably a single dose is at least 0.1× a typical full human dose, especially at least 0.25× a typical full human dose, in particular at least 0.5× a typical full human dose. Desirably the single dose is less than or equal to a full human dose. For example, the single dose may be 0.1 to 1× a typical full human dose, i.e. comprising 0.68 to 8.4 mg of squalene.

Particular single doses of interest include 0.1× a typical full human dose i.e. comprising 0.68 to 0.84 mg of squalene, 0.125× a typical full human dose i.e. comprising 0.85 to 1.1 mg of squalene, 0.25× a typical full human dose i.e. comprising 1.7 to 2.1 mg of squalene, such as 0.5× a typical full human dose i.e. comprising 3.4 to 4.2 mg of squalene or 1× a typical full human dose i.e. comprising 6.8 to 8.4 mg of squalene.

Squalene emulsion adjuvant derived from SEA160 may also include in particular a phosphate buffered saline, such as modified phosphate buffered saline.

The squalene emulsion adjuvant may be derived from SEAS44. Consequently, the squalene emulsion adjuvant may comprise squalene, tocopherol, polysorbate 80 and water. The squalene emulsion adjuvant may consist essentially of squalene, tocopherol, polysorbate 80 and water. Optionally the aqueous phase may contain additional components as desired or required depending upon the intended final presentation and vaccination strategy, such as buffers and/or tonicity modifying agents. Suitable buffers include Na₂HPO₄ and KH₂PO₄. Suitable tonicity modifying agents include NaCl and KCl. Modified phosphate buffered saline may be used, such as comprising Na₂HPO₄ and KH₂PO₄, NaCl and KCl.

Any of the α, γ, γ, δ, ε or ξ tocopherols can be used, but α-tocopherol is typically used. D-alpha-tocopherol and D/L-alpha-tocopherol can both be used. A particularly desirable alpha-tocopherol is D/L-alpha-tocopherol.

Typically, the weight ratio of squalene to tocopherol is 2.6 to 4.5, especially 2.8 to 4.3, in particular 3.25 to 4, such as 3.4 to 3.8 e.g. 3.6. Suitably the tocopherol is alpha-tocopherol, especially D/L-alpha-tocopherol.

Typically, the weight ratio of squalene to polysorbate 80 is 1.3 to 2.5, especially 1.56 to 2.3, in particular 1.75 to 2.15 such as 1.85 to 2 e.g. 1.94.

A single dose, such as a typical full human dose, of squalene emulsion adjuvant derived from SEAS44 may comprise 11.7 to 14.3 mg of squalene, such as 12.3 to 13.7 mg, in particular 13 mg. Higher or lower doses of squalene emulsion adjuvant derived from SEAS44 may be used. Suitably a single dose is at least 0.1× a typical full human dose, especially at least 0.25× a typical full human dose, in particular at least 0.5× a typical full human dose. Desirably the single dose is less than or equal to a full human dose. For example, the single dose may be 0.1 to 1× a typical full human dose, i.e. comprising 1.1 to 14.3 mg of squalene.

Particular single doses of interest include 0.1× a typical full human dose i.e. comprising 1.1 to 1.5 mg of squalene, 0.125× a typical full human dose i.e. comprising 1.4 to 1.8 mg of squalene, 0.25× a typical full human dose i.e. comprising 2.9 to 3.6 mg of squalene, such as 0.5× a typical full human dose i.e. comprising 5.8 to 7.2 mg of squalene or 1× a typical full human dose i.e. comprising 11.7 to 14.3 mg of squalene.

Squalene emulsion adjuvant derived from SEAS44 may also include in particular a phosphate buffered saline, such as modified phosphate buffered saline.

Self-emulsifying adjuvants, such as SEA160, SEAS44 and squalene emulsion adjuvant adjuvants derived therefrom, may be provided in dry form. For example, such dry self-emulsifying adjuvants may consist essentially of squalene and surfactant(s), such as in the case of SEA160 derived squalene emulsion adjuvants. Such dry self-emulsifying adjuvants may consist essentially of squalene and surfactant(s) or consist essentially of squalene, tocopherol and surfactant(s), such as in the case of SEAS44 derived tocopherol containing squalene emulsion adjuvants.

High pressure homogenization (HPH or microfluidisation) may be applied to yield squalene emulsion adjuvants which demonstrate uniformly small droplet sizes and long-term stability (see EP 0 868 918 B1 and WO2006/100109). Briefly, oil phase composed of squalene and tocopherol may be formulated under a nitrogen atmosphere. Aqueous phase is prepared separately, typically composed of water for injection or phosphate buffered saline, and polysorbate 80. Oil and aqueous phases are combined, such as at a ratio of 1:9 (volume of oil phase to volume of aqueous phase) before homogenisation and microfluidisation, such as by a single pass through an in-line homogeniser and three passes through a microfluidiser (at around 15000 psi). The resulting emulsion may then be sterile filtered, for example through two trains of two 0.5/0.2 um filters in series (i.e. 0.5/0.2/0.5/0.2), see WO2011/154444. Operation is desirably undertaken under an inert atmosphere, e.g. nitrogen. Positive pressure may be applied, see WO2011/154443.

WO2015/140138, WO2016/135154, WO2020/160080, Shah, 2014 Shah, 2015, Shah, 2019, and Lodaya, 2019 describe squalene emulsion adjuvants which are self-emulsifying adjuvant systems (SEAS) and their manufacture.

### Human Subjects

The subject may be of any age. In one embodiment the subject is a human infant (up to 12 months of age). In one embodiment the subject is a human child (less than 18 years of age). In one embodiment the subject is an adult human (aged 18-64). In one embodiment the subject is an older human (aged 65 or greater).

Doses (of coronavirus spike antigen and/or of squalene emulsion adjuvant), administered to younger children, such as less than 12 years of age, may be reduced relative to an equivalent adult dose, such as by 50%.

The methods of the invention are suitably intended for prophylaxis of coronavirus infection, such as SARS-CoV-2 infection, i.e. for administration to a subject which is not infected with a second coronavirus (by which is meant the ‘second coronavirus’ of the invention), e.g. SARS-CoV-2, such as not infected with a coronavirus.

In other embodiments the methods of the invention may be intended for treatment, e.g. for the treatment of coronavirus infection, such as SARS-CoV-2 infection, i.e. for administration to a subject which is infected with a coronavirus (such as infected with SARS-CoV-2), such as infected with a second coronavirus (such as infected with SARS-CoV-2).

In some embodiments the subject is a naïve subject i.e. a subject which has not previously been infected with or vaccinated against (e.g. not vaccinated against) a second coronavirus, such as infected with or vaccinated against (e.g. not vaccinated against) SARS-CoV-2, the subject may not have been infected with or vaccinated against (e.g. not vaccinated against) a coronavirus.

In other embodiments the subject is a primed subject i.e. a subject which has previously been infected with or vaccinated against (e.g. vaccinated against) a coronavirus (e.g. SARS-CoV-2), such as infected with or vaccinated against (e.g. vaccinated against) a second coronavirus (e.g. SARS-CoV-2).

Suitably, a primed subject was infected or vaccinated (e.g. vaccinated against) against a coronavirus (e.g. SARS-CoV-2), such as infected with or vaccinated against (e.g. vaccinated against) a second coronavirus (e.g. SARS-CoV-2), within 5 years of administration, such as within 2 years of administration, especially within 1 year of administration.

Those skilled in the art will appreciate that administration may be part of a multidose regime. In such cases, references to naïve and primed are to be taken as referring to the position prior to the first dose of the multidose regime.

In some embodiments the subject has previously been vaccinated with a coronavirus spike antigen (such as derived from a second coronavirus) in conjunction with a squalene emulsion adjuvant.

As used herein, the terms “treat” and “treatment” as well as words stemming therefrom, are not meant to imply a “cure” of the condition being treated in all individuals, or 100% effective treatment in any given population. Rather, there are varying degrees of treatment which one of ordinary skill in the art recognizes as having beneficial therapeutic effect(s). In this respect, the methods and uses herein can provide any level of treatment of coronavirus infection and, in particular, MERS-CoV, SARS-CoV-1, or SARS-CoV-2 related disease in a subject in need of such treatment, and may comprise reduction in the severity, duration, or number of recurrences over time, of one or more conditions or symptoms of coronavirus (e.g., MERS-CoV, SARS-CoV-1, or SARS-CoV-2) infection, and in particular SARS-CoV-2 related disease (e.g., COVID-19).

As used herein, “therapeutic immunization” or “therapeutic vaccination” refers to administration of the immunogenic compositions of the invention to a subject, who is known to be infected with a coronavirus (e.g., a betacoronavirus such as MERS-CoV, SARS-CoV-1, and/or SARS-CoV-2) at the time of administration, to treat the infection or pathogen-related disease or to prevent reinfection or reactivation. As used herein, “prophylactic immunization” or “prophylactic vaccination” refers to administration of the immunogenic compositions of the invention to a subject, within whom a coronavirus cannot be detected (e.g., who is not infected with coronavirus) at the time of administration, to prevent infection or coronavirus-related disease.

### First and Second Coronaviruses

Different coronaviruses may have identical spike proteins. Also, even if differing in spike protein sequences, coronaviruses may nevertheless be immunologically comparable or may be immunologically distinguishable.

By the term immunologically comparable in reference to two coronaviruses is meant that in convalescent sera from a subject (typically a human subject, although animal models such as non-human primates may alternatively be utilised) infected by one coronavirus the level of spike protein specific antibodies for said coronavirus as determined by ELISA is less than 2-fold different from the level of spike protein specific antibodies for the other coronavirus. Suitably the level of neutralising antibodies in convalescent sera for one coronavirus is less than 2-fold different from the level of neutralising antibodies for the other coronavirus.

By the term immunologically distinguishable in reference to two coronaviruses is meant that in convalescent sera from a subject (typically a human subject, although animal models such as non-human primates may alternatively be utilised) infected by one coronavirus the level of spike protein specific antibodies for said coronavirus as determined by ELISA is 2-fold or greater different (such as 5-fold or greater, especially 10-fold or greater, in particular 100-fold or greater) from the level of spike specific antibodies for the other coronavirus. Suitably the level of neutralising antibodies in convalescent sera for one coronavirus is 2-fold or greater different (such as 5-fold or greater, especially 10-fold or greater, in particular 100-fold or greater) from the level of neutralising antibodies for the other coronavirus.

Neutralisation may be determined by testing undertaken with the coronaviruses, or may be based on pseudo-virus testing (e.g. Lenti or VSV (vesicular stomatitis virus) expressing the relevant coronavirus spike proteins).

The first and second coronaviruses will typically be immunologically distinguishable. In some embodiments the level of spike protein specific antibodies in convalescent sera from a subject (typically a human subject, although animal models such as non-human primates may alternatively be utilised) infected by the first coronavirus is 2-fold to 10-fold, 10 to 100-fold or 100 to 1000-fold different from the level of spike specific antibodies for the second coronavirus. Suitably the level of neutralising antibodies in convalescent sera for the first coronavirus is 2-fold to 10-fold, 10 to 100-fold or 100 to 1000-fold different from the level of neutralising antibodies for the second coronavirus.

In some embodiments the first and second coronaviruses are alpha coronaviruses. In some embodiments the first and second coronaviruses are beta coronaviruses. In some embodiments the first and second coronaviruses are gamma coronaviruses. In some embodiments the first and second coronaviruses are delta coronaviruses.

In some embodiments the first and second coronaviruses are beta A coronaviruses, such as SARS beta A coronaviruses. In some embodiments the first and second coronaviruses are beta B coronaviruses, such as SARS beta A coronaviruses. In some embodiments the first and second coronaviruses are beta C coronaviruses, such as SARS beta C coronaviruses. In some embodiments the first and second coronaviruses are beta D coronaviruses, such as SARS beta D coronaviruses.

In some embodiments the first coronavirus is a MERS-CoV. In some embodiments the first coronavirus is a SARS-CoV-1. In some embodiments the first coronavirus is a SARS-CoV-2.

In some embodiments the second coronavirus is a MERS-CoV. In some embodiments the second coronavirus is a SARS-CoV-1. In some embodiments the second coronavirus is a SARS-CoV-2.

### Coronavirus Spike Antigen

Coronaviral infections initiate with binding of virus particles to host surface cellular receptors. Receptor recognition is therefore an important determinant of the cell and tissue tropism of the virus. In addition, the virus must be able to bind to the receptor counterparts in other species for inter-species-transmission to occur. With the exception of HCoV-OC43 and HKU1, both of which engage sugars for cell attachment, human coronaviruses (HCoVs) recognize proteinaceous receptors. HCoV-229E binds to human aminopeptidase N (hAPN); MERS-CoV interacts with human dipeptidyl peptidase 4 (hDPP4 or hCD26); and all three of SARS-CoV-1, hCoV-NL63, and SARS-CoV-2 interact with human angiotensin-converting enzyme 2 (hACE2). (Wang, 2020)

Structural proteins are encoded by one-third of coronavirus (CoV) genomes (one-third from the 3′ end), such structural proteins including the spike (S) glycoprotein, small envelope protein (E), integral membrane protein (M), and genome-associated nucleocapsid protein (N). Some coronaviruses also contain a hemagglutinin esterase (HE). Interspersed between these genes, are several genes coding for accessory proteins, many of which are involved in regulating the host immune system. The proteins E, M, and N are mainly responsible for the assembly of the virions, while the S protein has an essential role in virus entry and determines tissue and cell tropism, as well as host range. (Wang, 2016)

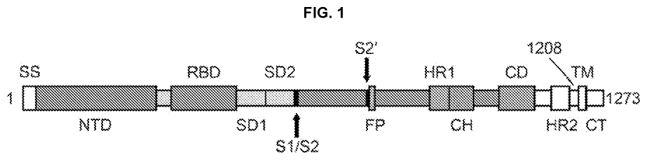

The process for coronavirus entry into host cells is mediated by the densely glycosylated, envelope-embedded, surface-located spike (S) glycoprotein (“S protein”), the SARS-CoV-2 spike being represented in FIG. 1. The S protein is a homotrimeric class I fusion protein with two subunits in each spike monomer (or “protomer”), called “S1” and “S2”, which are responsible for receptor recognition and membrane fusion, respectively. (Wrapp, 2020). The S protein is in a metastable prefusion conformation that, when triggered by the S1 subunit binding to a host cell receptor, undergoes a substantial structural rearrangement to fuse the viral membrane with the host cell membrane. (Li, 2016; Bosch, 2003; Wrapp, 2020; Wang, 2020). Receptor binding destabilizes the prefusion homotrimer, resulting in the shedding of the S1 subunit and transition of the S2 subunit to a stable postfusion conformation (in the case of MERS-CoV and SARS-CoV-2, but not SARS-CoV-1, the S protein is cleaved by host proteases (furin) into the S1 and S2 subunits, enabling S2 to form its stable postfusion conformation). (Wrapp, 2020; Wang, 2020; Follis, 2006). The S1 subunit can be further divided into an N-terminal domain (NTD) and a Receptor Binding Domain (RBD) (the RBD is also called a C-terminal domain (CTD)). (see Wrapp, 2020 and Wang, 2020 for the structures of SARS-CoV-1 and SARS-CoV-2; see Yuan, 2017 for the structures of MERS-CoV and SARS-CoV-1. hCoV-NL63, SARS-CoV-1, and SARS-CoV-2 all utilize the RBD to interact with the hACE2 receptor. (Wang, 2020)

According to the present invention, the squalene emulsion adjuvants are to be utilised in conjunction with a coronavirus spike antigen.

By the term ‘antigen’ is meant a polypeptide which is capable of eliciting an immune response in a subject. Suitably the immune response is a protective immune response, e.g. reducing partially or completely the severity of infection, such as reducing partially or completely the level of one or more symptoms and/or the time over which one or more symptoms are experienced by a subject, reducing the likelihood of developing an established infection after challenge (‘protection against infection’) and/or slowing progression of an associated illness (e.g. increasing or extending survival).

Suitably the antigen comprises at least one B or T cell epitope, suitably an antigen comprises B and T cell epitopes. The elicited immune response may be an antigen specific B cell response which produces neutralizing antibodies. The elicited immune response may be an antigen specific T cell response, which may be a systemic and/or a local response. The antigen specific T cell response may comprise a CD4+ T cell response, such as a response involving CD4+ T cells expressing a plurality of cytokines, e.g. IFNgamma, TNFalpha and/or IL2. Alternatively, or additionally, the antigen specific T cell response comprises a CD8+ T cell response, such as a response involving CD8+ T cells expressing a plurality of cytokines, e.g., IFNgamma, TNFalpha and/or IL2.

In some embodiments the coronavirus spike antigen comprises an epitope corresponding to residues 333, 334, 335, 336, 337, 339, 340, 341, 343, 344, 345, 346, 354, 356, 357, 358, 359, 360, 361, 440, 441 and 509 of SEQ ID NO: 1. (Pinto, 2020; Cathcart 2021) In some the coronavirus spike antigen comprises a variant epitope wherein residues corresponding to positions 333, 334, 335, 336, 337, 339, 340, 341, 343, 344, 345, 346, 354, 356, 357, 358, 359, 360, 361, 440, 441 and 509 of SEQ ID NO: 1 have at least 90% such as at least 95% identity to SEQ ID NO: 1.

Suitably, the coronavirus spike antigen comprises a RBD.

In some embodiments the amino acid sequence of the RBD domain of the first coronavirus has at least 90% identity to the RDB domain of the second coronavirus, such as at least 92% identity, especially at least 94% identity, in particular at least 96% identity, for example at least 98% identity.

In some embodiments the coronavirus spike antigen comprises, such as consists of, the sequence of the second coronavirus RBD domain. In other embodiments the coronavirus spike antigen comprises, such as consists of, a variant of the second coronavirus RBD domain having an amino acid sequence at least 90% identity thereto, such as at least 92% identity, especially at least 94% identity, in particular at least 96% identity, for example at least 98% identity.

In some embodiments the coronavirus spike antigen comprises, such as consists of, the sequence of SEQ ID NO: 3. In other embodiments the coronavirus spike antigen comprises, such as consists of, a variant of SEQ ID NO: 3 having at least 90% identity thereto, such as at least 92% identity, especially at least 94% identity, in particular at least 96% identity, for example at least 98% identity.

In some embodiments the coronavirus spike antigen comprises, such as consists of, the sequence of SEQ ID NO: 6. In other embodiments the coronavirus spike antigen comprises, such as consists of, a variant of SEQ ID NO: 6 having at least 90% identity thereto, such as at least 92% identity, especially at least 94% identity, in particular at least 96% identity, for example at least 98% identity.

In some embodiments the coronavirus spike antigen comprises, such as consists of, the sequence of SEQ ID NO: 8. In other embodiments the coronavirus spike antigen comprises , such as consists of, a variant of SEQ ID NO: 8 having at least 90% identity thereto, such as at least 92% identity, especially at least 94% identity, in particular at least 96% identity, for example at least 98% identity.

An RDB may be provided in a range of forms, for example, the coronavirus spike antigen may consist essentially of the RBD domain. For example, the coronavirus spike antigen may contain 1.1 times or fewer of the number of amino acid residues in the RBD domain fewer the coronavirus spike antigen.

An RBD may be provided as part of a larger coronavirus spike antigen, such as a full length coronavirus spike antigen, a CT-deleted coronavirus spike antigen or a TM-deleted coronavirus spike antigen. A “full length coronavirus spike antigen” herein means it comprises (from N-terminus to C-terminus) the NTD through to, and including, the cytoplasmic tail (CT). A “CT-deleted coronavirus spike antigen” herein means it comprises the NTD through to, and including, the transmembrane (TM) domain. A “TM-deleted coronavirus spike antigen” means it comprises the NTD up to, and excluding, the TM domain (but a TM-deleted coronavirus spike antigen may be operably linked at the C-terminus to a cytoplasmic tail or other (optionally heterologous) amino acid(s)).

In the context of administration of a coronavirus spike antigen, it is desirable to deliver a prefusion conformation coronavirus spike antigen. Sequence alternations may therefore be introduced to favour or lock a coronavirus spike antigen in prefusion conformation, such as one or more proline substitutions, preferably one or two proline substitutions, and introduced at or near (e.g., within two residues N- or C-terminal to, or within two residues C-terminal to) the boundary between the Heptad Repeat 1 (HR1) and the Central Helix (CH). The HR1/CH boundary within SARS-CoV-2 sequence SEQ ID NO: 1 is between D985 and K986 (see Wrapp, 2020). To lock SARS-CoV-2 S protein in prefusion conformation, it is sufficient to introduce one proline residue. In particular, it is sufficient to substitute K986, numbered according to SEQ ID NO: 1, with proline (P). Therefore, a preferred embodiment utilises a modified coronavirus spike antigen comprising a proline (P) at the residue corresponding to 986 of the sequence SEQ ID NO: 1. It was previously demonstrated that the introduction of two proline residues at or near the boundary between the SARS-CoV-2 S protein HR1 and CH is sufficient to lock the S protein in prefusion conformation (see WO2018/081318; Graham, 2020; Wrapp, 2020). In particular, the substitution of both K986 and V987, numbered according to SEQ ID NO: 1, to proline was shown to lock SARS-CoV-2 S protein in prefusion conformation (WO2018/081318; Graham, 2020; Wrapp, 2020). Therefore, another embodiment utilises a modified coronavirus spike antigen comprising the mutation of two immediately adjacent residues at or within two residues of the HR1/CH boundary wherein the mutations are substitutions to proline. A further embodiment utilises a modified coronavirus spike antigen comprising prolines (P) at the residues corresponding to 986 and 987 of the sequence SEQ ID NO: 1.

To provide a prefusion coronavirus spike antigen or to promote the formation of trimeric complexes, it may be desirable to insert a trimerization domain (e.g., the T4 fibritin trimerization (foldon) motif) into the C-terminus. In particular, a coronavirus spike antigen having an inactive transmembrane domain (e.g., inactive by deletion) or, optionally, lacking the entire C-terminus (e.g., lacking by deletion), comprises the ectodomain sequence operably linked (e.g., through the inclusion of one or more linker residues) to a trimerization domain sequence (e.g., a heterologous trimerization domain) such as the T4 fibritin trimerization (foldon) motif (see an example of this technique with MERS-CoV and SARS-CoV-1 by Yuan, 2017).

It may be desirable to keep the S1 and S2 subunits operably linked, especially if prefusion conformation is desired. In the context of MERS-CoV or SARS-CoV-2 S proteins, it is thus desirable to prevent furin cleavage of the S1 and S2 subunits. For betacoronavirus delivery of a MERS-CoV or SARS-CoV-2 coronavirus spike antigen, it is therefore desirable to deliver a furin-cleavage abrogated coronavirus spike antigen. Furin-cleavage abrogation may be achieved by introducing substitution mutations into the R-X-X-R furin recognition/cleavage motif (where the arginines (R) are “furin motif arginines” and where X is any amino acid) as was previously shown for the ⁶⁸²RRAR⁶⁸⁵ SARS-CoV-2 S1/52 furin recognition site (see Wrapp, 2020) and for the ⁷³⁰RSVR⁷³³MERS-CoV S1/52 furin recognition site, corresponding to residues 748 to 751 of SEQ ID NO: 8 (see Millet, 2014). Yuan, 2017 also demonstrates a furin abrogated MERS-CoV S protein by mutation within the furin recognition motif. It is notable that wild type SARS-CoV-1 S protein maintains the residue corresponding to the C-terminal furin motif arginine (R), not the N-terminal furin motif arginine (see Wrapp, 2020). In particular, furin-cleavage abrogation may be achieved by introducing one or more substitution mutations into the furin motif, wherein the one or more substitution mutations comprise a substitution of one or both of the furin motif arginines (R). An embodiment therefore utilises a coronavirus spike antigen comprising one or more substitution mutations at the residues corresponding to R682 to R685 of the sequence SEQ ID NO: 1, wherein the one or more substitution mutations include the substitution of one or both of the residues corresponding to R682 and R685 of the sequence SEQ ID NO: 1; optionally wherein the wild type or control coronavirus spike antigen is cleaved by furin (e.g., MERS-CoV or SARS-CoV-2 S protein). In certain embodiments an RRAR motif may, for example, be replaced with GSAS or SGAG.

Antibody-dependent enhancement (ADE) of viral infection or disease may be a concern (see Tirado, 2003). ADE has been observed for coronaviruses (Wan, 2020; Walls, 2019). One approach to reduce the risk of ADE in the context of vaccination by delivering an antigen to a subject, is to introduce receptor binding mutations into the antigen sequence. Where the antigen is a modified coronavirus spike antigen, wherein its wild type counterpart binds hACE2 as receptor (e.g., hCoV-NL63, SARS-CoV-1, and/or SARS-CoV-2), it may therefore be desirable for the antigen sequence to comprise one or more receptor binding mutations (e.g., receptor binding knock-down mutations, receptor binding knock-out mutations, or receptor binding glycan mutations) to avoid eliciting antibodies that are comparable to hACE2 and thereby avoid, for example, enhancing the possibility of triggering conformational changes from pre- to postfusion S protein during the course of natural infection. The RBDs of at least SARS-CoV-1 and SARS-CoV-2 have already been characterized and compared, providing identification of corresponding residues (Tai, 2020). Certain embodiments utilise a modified coronavirus spike antigen (e.g., hCoV-NL63, SARS-CoV-1, and/or SARS-CoV-2 S protein or fragment thereof) with an amino acid sequence comprising a receptor binding mutation.

Optionally, to facilitate expression and recovery, the coronavirus spike antigen may include a signal peptide at the N-terminus. A signal peptide can be selected from among numerous signal peptides known in the art and is typically chosen to facilitate production and processing in a system selected for recombinant expression. In one embodiment, the signal peptide is the one naturally present in the native viral spike protein (see, e.g., SEQ ID NO: 1). In another embodiment, the signal peptide is a Gaussian Luciferase signal sequence, a human CD5 signal sequence, a human CD33 signal sequence, a human IL2 signal sequence, a human IgE signal sequence, a human Light Chain Kappa signal sequence, a JEV short signal sequence, a JEV long signal sequence, a Mouse Light Chain Kappa signal sequence, a SSP signal sequence, or a Gaussian Luciferase (AKP). As used herein, a “mature” sequence means it lacks the N-terminal signal sequence (signal peptide). A coronavirus spike antigen may contain the signal peptide, or may be in a mature form wherein the signal peptide has been cleaved.

A coronavirus spike antigen may comprise heterologous amino acid residues, such as one or more tags to facilitate detection (e.g. an epitope tag for detection by monoclonal antibodies) and/or purification (e.g. a polyhistidine-tag to allow purification on a nickel-chelating resin) of the protein or fragment. In a certain embodiment, the sequence further comprises a cleavable linker. A cleavable linker allows for the tag to be separated, for example, by the addition of an agent capable of cleaving the linker. A number of different cleavable linkers are known to those of skill in the art.

In certain embodiments it may thus be necessary to truncate the ectodomain, so certain embodiments utilize a modified betacoronavirus S protein fragment having a truncated ectodomain that lacks 1, 2, 3, 4, 5, 6, 7, 8, 9, 10, 11, 12, 13, 14, 15, 16, 17, 18, 19 or 20 amino acid residues of the natural ectodomain.

A coronavirus spike antigen with an inactive transmembrane domain (e.g., inactive by having a truncated TM domain (“TM-truncated”, such as a deleted TM domain “TM-deleted”) cannot reside within a lipid bilayer and may, therefore, be more easily purified and at higher yield. It may be desirable to increase the solubility of a coronavirus spike antigen by, for example, providing a TM-inactive (e.g., TM-truncated or TM-deleted) coronavirus spike antigen. In certain embodiments a TM-truncated coronavirus spike antigen is utilised that is operably linked at its C-terminus to a heterologous amino acid sequence (such as a cytoplasmic tail (CT)).

In certain embodiments a coronavirus spike antigen has a truncated cytoplasmic domain.

“Fragment,” refers to a portion (that is, a subsequence) of a polypeptide and is generated by cleaving one or more residues from either end of the reference polynucleotide/polypeptide sequence (e.g., deletion of the transmembrane domain). In this way, a fragment is an exemplary deletion coronavirus spike antigen. A fragment is typically at least 100, 200, 300, 400, 500, 600, 700, 800, 900, 1000, or 1100 amino acids in length (and any integer value in between). An “immunogenic fragment” of an antigen is a portion of a polypeptide that elicits an immune response. An “immunogenic fragment” refers to a molecule containing one or more epitopes (e.g., linear, conformational or both) capable of stimulating a host's immune system to make a humoral and/or cellular antigen-specific immunological response (i.e. an immune response which specifically recognizes a naturally occurring polypeptide, i.e. full length coronavirus spike antigen). An immunogenic fragment of an antigen retains at least one immunogenic epitope of its reference (“source”) polypeptide. An “epitope” is that portion of an antigen that determines its immunological specificity. T- and B-cell epitopes can be identified empirically (e.g. using PEPSCAN or similar methods). Herein, when the reference (“source”) polypeptide is described as having one or more specific amino acid substitutions, it is meant that a “fragment thereof” also comprises that one or more specific amino acid substitutions. An exemplary immunogenic fragment for use herein consists a coronavirus spike protein Receptor Binding Domain (RBD), such as an immunogenic fragment comprising the amino acids corresponding to residues of SEQ ID No. 3, optionally linked (directly or indirectly) to additional coronavirus spike residues or to a pharmaceutically acceptable carrier (e.g. a nanoparticle or IgG1 Fc). Such immunogenic fragments consisting of a spike protein RBD were previously described for candidate MERS-CoV and SARS-CoV-1 vaccines (including Fc chimeric proteins) (Zheng, 2008; Du, 2009; Wang, 2016).

Suitably a sequence comprising the coronavirus spike antigen contains 3000 residues or fewer, especially 2000 residues or fewer, in particular 1800 residues or fewer, such as 1500 residues or fewer. The coronavirus spike antigen may contain 1300 residues or fewer, 1200 residues or fewer, 1000 residues or fewer, 800 residues or fewer, 600 residues or fewer, 400 residues or fewer, 250 residues or fewer or 200 residues or fewer.

Suitably the coronavirus spike antigen contains 100 residues or more, especially 110 residues or more, in particular 120 residues or more, such as 150 residues or more.

Suitably a sequence comprising the coronavirus spike antigen contains 100 to 3000 residues, especially 100 to 1500 residues, in particular 150 to 1200 residues.

A coronavirus spike antigen of use in the present invention may comprise a fragment or variant of a native coronavirus protein which is capable of eliciting neutralising antibodies and/or a T cell response (such as a CD4 or CD8 T cell response) to a coronavirus, suitably a protective immune response.

A SARS-CoV-2 spike antigen of use in the present invention may comprise, such as consists of, a fragment or variant of a native SARS-CoV-2 S protein which is capable of eliciting neutralising antibodies and/or a T cell response (such as a CD4 or CD8 T cell response) to SARS-CoV-2, suitably a protective immune response.

A SARS-CoV-2 spike antigen may comprise, such as consist of, a full length S protein (such as SEQ ID NO:1). Alternatively, a SARS-CoV-2 spike antigen may comprise, such as consist of, an amino acid sequence having at least 90% identity to the amino acid sequence set forth in SEQ ID NO:1. A SARS-CoV-2 spike antigen may comprise, such as consist of, an amino acid sequence having at least 95% identity to the amino acid sequence set forth in SEQ ID NO:1, especially at least 98% identity to the amino acid sequence set forth in SEQ ID NO:1, in particular at least 99% identity to the amino acid sequence set forth in SEQ ID NO:1, such as 100% identity to the amino acid sequence set forth in SEQ ID NO:1.

A SARS-CoV-2 spike antigen may comprise, or consist of, one or more domains of a full length SARS-CoV-2 S protein, such as the ectodomain (SEQ ID NO:2) or receptor binding domain (RBD, SEQ ID NO:3), or variants thereof.

A SARS-CoV-2 spike antigen may comprise, such as consist of, an amino acid sequence having at least 90% identity to the amino acid sequence set forth in SEQ ID NO:2. A SARS-CoV-2 spike antigen may comprise, such as consist of, an amino acid sequence having at least 95% identity to the amino acid sequence set forth in SEQ ID NO:2, especially at least 98% identity to the amino acid sequence set forth in SEQ ID NO:2, in particular at least 99% identity to the amino acid sequence set forth in SEQ ID NO:2, such as 100% identity to the amino acid sequence set forth in SEQ ID NO:2.

A SARS-CoV-2 spike antigen may comprise, such as consist of, an amino acid sequence having at least 90% identity to the amino acid sequence set forth in SEQ ID NO:3. A SARS-CoV-2 spike antigen may comprise, such as consist of, an amino acid sequence having at least 95% identity to the amino acid sequence set forth in SEQ ID NO:3, especially at least 98% identity to the amino acid sequence set forth in SEQ ID NO:3, in particular at least 99% identity to the amino acid sequence set forth in SEQ ID NO:3, such as 100% identity to the amino acid sequence set forth in SEQ ID NO:3.

Suitably a SARS-CoV-2 spike antigen is pre-fusion stabilised to facilitate appropriate presentation to the immune system. For example, Wrapp and colleagues (Wrapp et al., 2020) produced a recombinant prefusion S ectodomain using a stabilization strategy that proved effective for other betacoronavirus S proteins (Pallesen et al, 2017; Kirchdoerfer et al, 2018). To this end, starting with the SARS-CoV-2 polynucleotide sequence (GenBank accession number MN908947.3), a gene encoding residues 1 to 1208 of SARS-CoV-2 S protein (UniProt accession number P0DTC2 version 1 dated 22 Apr. 2020) with proline substitutions at residues 986 and 987, a “GSAS” substitution at the furin cleavage site (residues 682 to 685) a C-terminal T4 fibritin trimerization motif, an HRV3C protease cleavage site, a TwinStrepTag and an 8×HisTag was synthesized and cloned into the mammalian expression vector pαH. Purification tags such as a HisTag or TwinStrepTag would generally be avoided in commercial vaccines, therefore if present during expression would typically be subsequently removed during later processing.

Residues 1 to 1208 of SARS-CoV-2 S protein with proline substitutions at residues 986 and 987, a “GSAS” substitution at the furin cleavage site are provided in SEQ ID NO:4, which is an example of a pre-fusion stabilized ectodomain of SARS-CoV-2 S protein.

A SARS-CoV-2 spike antigen may comprise, such as consist of, an amino acid sequence having at least 90% identity to the amino acid sequence set forth in SEQ ID NO:4. A SARS-CoV-2 spike antigen may comprise, such as consist of, an amino acid sequence having at least 95% identity to the amino acid sequence set forth in SEQ ID NO:4, especially at least 98% identity to the amino acid sequence set forth in SEQ ID NO:4, in particular at least 99% identity to the amino acid sequence set forth in SEQ ID NO:4, such as 100% identity to the amino acid sequence set forth in SEQ ID NO:4.

Suitably a SARS-CoV-2 spike antigen is a pre-fusion stabilised spike antigen.

In one embodiment, the SARS-CoV-2 spike antigen is the stabilized recombinant prefusion S ectodomain disclosed by Wrapp et al., 2020.

A SARS-CoV-2 spike antigen (such as a pre-fusion stabilized SARS-CoV-2 S protein) may desirably be in the form of a trimer and consequently may comprise a trimerization motif, such as a T4 fibritin trimerization motif, more suitably a C-terminal T4 fibritin trimerization motif. Alternative trimerization motifs include, for example, a domain derived from collagen called ‘Trimer-Tag’ such as disclosed in Liu et al., 2017, or a molecular clamp, such as that disclosed in WO2018/176103.

In some embodiments the coronavirus spike antigen comprises, such as consists of, a prefusion stabilised spike having a mutated S1/S2 furin cleavage site, deleted transmembrane and cytoplasmic region and incorporating a T4-foldon trimerization domain, such as described in Francica, 2021. The coronavirus spike antigen may be derived from the Wuhan YP_009724390.1 strain S sequence.

In some embodiments the coronavirus spike antigen comprises, such as consists of, an RBD domain and wherein the RBD domain is displayed on a nanoparticle. The RBD domain may be genetically fused to the N terminus of a nanoparticle component (e.g. trimeric I53-50A) using linkers, such as of 8, 12 or 16 glycine and serine residues, such as described in Walls, 2020. The coronavirus RBD domain may be derived from the Wuhan YP_009724390.1 strain S sequence.

In some embodiments the coronavirus spike antigen comprises, such as consists of, a full-length prefusion stabilised spike and wherein the full-length prefusion stabilised spike is displayed on a nanoparticle. The coronavirus spike antigen may be derived from the Wuhan YP_009724390.1 strain S sequence.

In some embodiments the coronavirus spike antigen comprises, such as consists of, a prefusion stabilised trimer, such as an S-Trimer as described Richmond, 2021. The coronavirus spike antigen may be derived from the Wuhan YP_009724390.1 strain S sequence.

In some embodiments the coronavirus spike antigen is presented in the form of a virus like particle.

Natural sequence variation exists between coronavirus S proteins, even between S proteins from the same virus. Known variations in SARS-Cov-2 S proteins include: L18F, Q52R, 69-70 deletion, A67V, D80A, 195I, R102I, G142V, Y144F, 144 deletion, H146Y, F157L, D215G, 242-244 deletion, R246I, D364Y, V367F, R408I, K417N, W436R, N439K, G446V, L452M, L452R, Y453F, L455F, L455Y, A475V, S477N, V483A, E484K, E484Q, G485R, F486L, F490L, F490S, Q493K, Q493N,S494P, Q498Y, N501T, N501Y, A570D, Q613H, D614G, Q677H, 1678I, S680F, P681H, P681R, A684V, A701V, T716I, D796H, F888L, A930V, D936Y, S982A, E111K and D1118H (Public Health England Technical Briefing 7, 11 Mar. 2021; Bakhshandeh, 2021; Greaney, 2021).

Despite the large number of sequence variations, to date only E484, in particular E484K, has been associated with a substantial impact on antibody binding and vaccination efficacy. It may be expected that selection pressure may lead to new escape mutants becoming important in the future. Mutations in the 443 to 450 loop, such as G446, in particular G446V, can cause a large drop in plasma antibody binding and neutralization (Greaney, 2021).

In some embodiments the first coronavirus comprises a spike sequence having an E484 mutation, such as E484K, and the second coronavirus comprises a spike sequence having E484.

In some embodiments the second coronavirus comprises a spike sequence having E484, and the second coronavirus comprises a spike sequence having an E484 mutation, such as E484K.

In some embodiments the first coronavirus comprises a spike sequence having an E484 mutation, such as E484K, and the coronavirus spike antigen comprises E484.

In some embodiments the first coronavirus comprises a spike sequence having E484, and the coronavirus spike antigen comprises an E484 mutation, such as E484K.

In some embodiments the second coronavirus is Wuhan YP_009724390.1 strain. In some embodiments the first coronavirus is B.1.351, B.1.525, B.1.1.318, R.1, R.2, B.1.1.28, P.1, P.2 or P.3.

A typical human dose of coronavirus spike antigen may be 1 to 100 μg, about 25 μg (such as 22.5 to 27.5 μg) or about 50 μg (such as 45 to 55 μg).

### Cross-Reactive Immune Response

An immune response is cross-reactive in that the coronavirus spike antigen from the second coronavirus can induce an antigen specific humoral and/or cellular immune response against the coronavirus spike antigen from the first coronavirus. In particular the level of spike protein specific antibodies, such as neutralising antibodies, to the first coronavirus may be increased by the methods of the invention.

While in the case of two immunologically distinguishable coronaviruses the level of spike protein specific antibodies in convalescent sera is 2-fold or greater different (such as 5-fold or greater, especially 10-fold or greater, in particular 100-fold or greater), the methods of the invention may provide a level of spike specific antibodies in immunised subjects which has a reduced difference. For example less than 100-fold different, such as less than 50-fold different, especially less than 20-fold different, in particular less than 10-fold different, e.g. less than 5-fold different, such as less than 2-fold different. In some embodiments the difference between spike protein specific antibodies (e.g. neutralising antibodies) for the first and second coronaviruses in immunised subjects is at least 1.5-fold, such as at least 2-fold, especially at least 5-fold, in particular at least 10-fold lower than the difference between spike protein specific antibodies for the first and second coronaviruses in convalescent sera (typically a human subject, although animal models such as non-human primates may alternatively be utilised). By way of illustration, in convalescent sera (e.g. non-human primate sera), if the ratio of neutralising antibodies is 10-fold different for the first and second coronaviruses and in immunised sera (e.g. non-human primate sera) the ratio of neutralising antibodies for the first and second coronaviruses is 5-fold different, then the effect of immunisation is a 2-fold lower level of difference.

### Sequence Alignments

Identity or homology with respect to a sequence is defined herein as the percentage of amino acid residues in the candidate sequence that are identical with the reference amino acid sequence after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)] can be used in conjunction with the computer program. For example, the percent identity can then be calculated as: the total number of identical matches multiplied by 100 and then divided by the sum of the length of the longer sequence within the matched span and the number of gaps introduced into the shorter sequences in order to align the two sequences.

“Sequence identity” as used herein means matches between two nucleic acids or two amino acids. As would be understood within the field, a “match” during sequence alignment is assigned when the two nucleic/amino acids are the same or comparable to the other (such as when one is a synthetic analog of the other). To be clear, as used herein a sequence “match”, and therefore “sequence identity”, does not encompass what are known as “conserved substitutions” or “conservatively substituted residues” by the field. Unless specified otherwise, “sequence identity” as used herein means the nucleic/amino acids are the same (identical) and not merely similar or “conserved substitutions” of each other. “Sequence identity” is determined by sequence alignment, such as by pairwise, global alignment using the Needleman-Wunsch algorithm and default parameters. Pairwise sequence alignment and the various algorithms therefor, is well understood in the art (Mullan 2005 Briefings in Bioinformatics 7(1):113-115); as are multiple sequence alignment methodologies and algorithms (Daugelaite et al. 2013 ISRN Biomathematics 2013 (Article ID 615630): 14 pages). As an example, Clustal Omega is a popular multiple sequence alignment (MSA) tool by EMBL-EBI and COBALT is a popular MSA tool by NCBI (each with its own functionalities). For clarification, N-terminal or C-terminal (or 5′ or 3′) residues such as signal peptides, tags, or leader sequences may be excluded from an alignment. With many alignment tools, an asterisk (*) denotes identity between residues, a colon (:) denotes highly similar residues, a period (.) denotes weakly similar residues, and a space ( ) denotes no similarity; a hyphen (−) denotes a gap. “Percent sequence identity” between two amino acid sequences or between two nucleic acid sequences means the percentage of nucleic/amino acid residue matches between the two sequences over the reported aligned region (including any gaps in the length); such as the percentage of identical residue matches between the two sequences over the reported aligned region following pairwise, global alignment using the Needleman-Wunsch algorithm and default parameters. It is well understood in the field that two sequences may be identical but-for one or more inserted or deleted residues (gaps). Such gaps may be “end gaps” (i.e., insertions or deletions at the N-terminal or C-terminal (for protein) or 5′ or 3′ (for polynucleotide) ends of the sequence) or “internal gaps” (gaps in the length of a sequence, i.e., are not located at the end (first or last residue) of the sequence). Therefore, use of an alignment algorithm that accounts for at least internal gaps is preferred. One such alignment algorithm is the pairwise, global Needleman-Wunsch algorithm. Percent sequence identity herein is preferably determined by pairwise, global alignment with the Needleman-Wunsch algorithm (Needleman and Wunsch, 1970 J. Mol. Biol. 48(3): 443-453), using default parameters (“Needleman-Wunsch algorithm with default parameters” means: Gap opening penalty (GAP OPEN)=10.0 and with Gap extension penalty (GAP EXTEND)=0.5, with no penalty for end Gaps (END GAP PENALTY=FALSE), and using the EBLOSUM62 scoring matrix (BLOSUM62 scoring table) for amino acid sequences or EDNAFULL scoring matrix for nucleotide sequences). The Needleman-Wunsch algorithm and these default parameters is implemented in the publicly available Needle tool in the EMBL-EBI EMBOSS package (Rice et al. 2000 Trends Genetics 16: 276-277; see also the World Wide Web at ebi.ac.uk/Tools/psa/emboss_needle). Preferably, the default “pair” output format from EMBOSS Needle is used. It may therefore be specified herein that “X has Y % sequence identity to the sequence SEQ ID NO: W, as determined by the Needleman and Wunsch algorithm with default parameters”. Percent sequence identity” is calculated by dividing the [total number of identical residues] (numerator) by the [total number of aligned residues] (denominator) and then multiplying that result by 100; optionally then rounding down to the next nearest whole number. It is notable that the denominator for a percent sequence identity calculation following alignment with the Needleman and Wunsch algorithm with default parameters may not be equal to the total length of either sequence.

### Additional Antigens

The present invention may involve a plurality of antigenic components (or polynucleotides encoding antigens), for example with the objective to elicit a broad immune response e.g. to a pathogen, such as a Coronavirus, or to elicit responses to multiple pathogens.

In some embodiments the invention utilises one coronavirus spike antigen. In some embodiments the invention utilises one coronavirus antigen, such as one antigen, which is the coronavirus spike antigen.

### Formulation and Administration

The coronavirus spike antigen and squalene emulsion adjuvant may be administered as a formulation containing the coronavirus spike antigen and squalene emulsion adjuvant (‘co-formulation’ or ‘co-formulated’). Alternatively the coronavirus spike antigen and squalene emulsion adjuvant may be administered as a first formulation containing the coronavirus spike antigen and a second formulation containing the squalene emulsion adjuvant (‘separate formulation’ or ‘separately formulated’). When separately formulated, the coronavirus spike antigen and squalene emulsion adjuvant may be administered through the same or different routes, to the same or different locations, and at the same or different times.

The coronavirus spike antigen and squalene emulsion adjuvant may be administered via various suitable routes, including parenteral, such as intramuscular or subcutaneous administration. The coronavirus spike antigen and squalene emulsion adjuvant may be administered via different routes. Suitably the coronavirus spike antigen and squalene emulsion adjuvant are administered via the same route, in particular intramuscularly.

When administered as separate formulations, the coronavirus spike antigen and squalene emulsion adjuvant are desirably administered to locations with sufficient spatial proximity such that the adjuvant effect is adequately maintained. For example, spatial proximity is sufficient to maintain at least 50%, especially at least 75% and in particular at least 90% of the adjuvant effect seen with administration at to the same location. The coronavirus spike antigen and squalene emulsion adjuvant are desirably administered to a location draining to the same lymph node, such as to the same limb, in particular to the same muscle.

Suitably the coronavirus spike antigen and squalene emulsion adjuvant are administered intramuscularly to the same muscle. In certain embodiments, the coronavirus spike antigen and squalene emulsion adjuvant are administered to the same location.

The spatial separation of administration locations may be at least 5 mm, such as at least 1 cm.

The spatial separation of administration locations may be less than 10 cm, such as less than 5 cm apart.

When administered as separate formulations, the coronavirus spike antigen and squalene emulsion adjuvant are desirably administered with sufficient temporal proximity such that the adjuvant effect is adequately maintained. For example, temporal proximity is sufficient to maintain at least 50%, especially at least 75% and in particular at least 90% of the adjuvant effect seen with administration at (essentially) the same time.

When administered as separate formulations, the coronavirus spike antigen and squalene emulsion adjuvant may be administered within 12 hours. Suitably the coronavirus spike antigen and squalene emulsion adjuvant are administered within 6 hours, especially within 2 hours, in particular within 1 hour, such as within 30 minutes and especially within 15 minutes (e.g. within 5 minutes).

When administered as separate formulations, the coronavirus spike antigen and squalene emulsion adjuvant may be administered within 84 hours, such as within 60 hours, especially within 36 hours, in particular within 24 hours. In one embodiment the coronavirus spike antigen and squalene emulsion adjuvant are administered within 12 to 36 hours. In another embodiment the coronavirus spike antigen and squalene emulsion adjuvant are administered within 36 to 84 hours.

The delay between administration of the coronavirus spike antigen and squalene emulsion adjuvant may be at least 5 seconds, such as 10 seconds, and in particular at least 30 seconds.

When administered as separate formulations, if the coronavirus spike antigen and squalene emulsion adjuvant are administered with a delay, the coronavirus spike antigen may be administered first and the squalene emulsion adjuvant administered second. Alternatively, the squalene emulsion adjuvant is administered first and the coronavirus spike antigen administered second. Appropriate temporal proximity may depend on the order of administration.

Desirably, the coronavirus spike antigen and squalene emulsion adjuvant are administered without intentional delay (accounting for the practicalities of multiple administrations).

In addition to co-formulated or separately formulated presentations of coronavirus spike antigen and squalene emulsion adjuvant for direct administration, the coronavirus spike antigen and squalene emulsion adjuvant may initially be provided in various forms which facilitate manufacture, storage and distribution. For example, certain components may have limited stability in liquid form, certain components may not be amendable to drying, certain components may be incompatible when mixed (either on a short- or long-term basis). Independent of whether coronavirus spike antigen and squalene emulsion are co-formulated at administration, they may be provided in separate containers the contents of which are subsequently combined. The skilled person will appreciate that many possibilities exist, although it is generally desirable to have a limited number of containers and limited number of required steps to prepare the final co-formulation or separate formulations for administration.

Coronavirus spike antigen may be provided in liquid or dry (e.g. lyophilised) form. The preferred form will depend on factors such as the precise nature of the coronavirus spike antigen, e.g. if the coronavirus spike antigen is amenable to drying, or other components which may be present. The coronavirus spike antigen is typically provided in liquid form.

The squalene emulsion adjuvant may be provided in liquid or dry form. The preferred form will depend on the precise nature of the squalene emulsion adjuvant, e.g. if capable of self-emulsification, and any other components present. The squalene emulsion adjuvant is typically provided in liquid form.

Typically a coronavirus spike antigen and squalene emulsion adjuvant are provided as a liquid co-formulation. A liquid co-formulation enables convenient administration at the point of use.

In other embodiments the coronavirus spike antigen and squalene emulsion adjuvant are provided as a dry co-formulation, the dry co-formulation being reconstituted prior to administration. A dry co-formulation, where the components of the formulation are amendable to such presentation, may improve stability and thereby facilitate longer storage.

The coronavirus spike antigen and squalene emulsion adjuvant may be provided in separate containers. The invention therefore provides a coronavirus spike antigen for use with a squalene emulsion adjuvant according to the present invention. Also provided is a squalene emulsion adjuvant for use with a coronavirus spike antigen according to the present invention. Further provided is a kit comprising:
(i) a first container comprising a coronavirus spike antigen; and
(ii) a second container comprising a squalene emulsion adjuvant, for use according to the present invention.

The coronavirus spike antigen may be in liquid form and the squalene emulsion adjuvant may be in liquid form. In such cases the contents of the first and second containers may be intended for combination to provide a co-formulation for administration. Alternatively, the contents of each container may be intended for separate administration as the first and second formulations.

The coronavirus spike antigen may be in dry form and the squalene emulsion adjuvant may be in liquid form. In such cases the contents of the first and second containers may be intended for combination to provide a co-formulation for administration. Alternatively, the coronavirus spike antigen may be intended to be reconstituted prior to the contents of each container being used for separate administration as the first and second formulations.

The squalene emulsion adjuvant may be in dry form and the coronavirus spike antigen may be in liquid form. In such cases the contents of the first and second containers may be intended for combination to provide a co-formulation for administration. Alternatively, the squalene emulsion adjuvant may be intended to be reconstituted prior to the contents of each container being used for separate administration as the first and second formulations.

The coronavirus spike antigen may be in dry form and the squalene emulsion adjuvant may be in dry form. In such cases the contents of the first and second containers may be intended for reconstitution and combination to provide a co-formulation for administration. Reconstitution may occur separately before combination, or the contents of one container may be reconstituted and then used to reconstitute the contents of the other container. Alternatively, the contents of the first and second containers may be intended for reconstitution prior to the contents of each container being used for separate administration as the first and second formulations.

If appropriate to the circumstances, liquid forms may be stored frozen.

The precise composition of liquid used for reconstitution will depend on both the contents of a container being reconstituted and the subsequent use of the reconstituted contents e.g. if they are intended for administration directly or may be combined with other components prior to administration. A composition (such as those containing coronavirus spike antigen or squalene emulsion adjuvant) intended for combination with other compositions prior to administration need not itself have a physiologically acceptable pH or a physiologically acceptable tonicity; a formulation intended for administration should have a physiologically acceptable pH and should have a physiologically acceptable osmolality.

The pH of a liquid preparation is adjusted in view of the components of the composition and necessary suitability for administration to the human subject. The pH of a formulation is generally at least 4, especially at least 5, in particular at least 5.5 such as at least 6. The pH of a formulation is generally 9 or less, especially 8.5 or less, in particular 8 or less, such as 7.5 or less. The pH of a formulation may be 4 to 9, especially 5 to 8.5, in particular 5.5 to 8, such as 6.5 to 7.4 (e.g. 6.5 to 7.1).

For parenteral administration, solutions should have a physiologically acceptable osmolality to avoid excessive cell distortion or lysis. A physiologically acceptable osmolality will generally mean that solutions will have an osmolality which is approximately isotonic or mildly hypertonic. Suitably the formulations for administration will have an osmolality of 250 to 750 mOsm/kg, especially 250 to 550 mOsm/kg, in particular 270 to 500 mOsm/kg, such as 270 to 400 mOsm/kg. Osmolality may be measured according to techniques known in the art, such as by the use of a commercially available osmometer, for example the Advanced® Model 2020 available from Advanced Instruments Inc. (USA).

Liquids used for reconstitution will be substantially aqueous, such as water for injection, phosphate buffered saline and the like. As mentioned above, the requirement for buffer and/or tonicity modifying agents will depend on the on both the contents of the container being reconstituted and the subsequent use of the reconstituted contents. Buffers may be selected from acetate, citrate, histidine, maleate, phosphate, succinate, tartrate and TRIS. The buffer may be a phosphate buffer such as Na/Na₂PO₄, Na/K₂PO₄ or K/K₂PO₄.

Suitably, the formulations used in the present invention have a dose volume of between 0.05 ml and 1 ml, such as between 0.1 and 0.6 ml, in particular a dose volume of 0.45 to 0.55 ml, such as 0.5 ml. The volumes of the compositions used may depend on the subject, delivery route and location, with smaller doses being given by the intradermal route or if both the coronavirus spike antigen and squalene emulsion adjuvant are delivered to the same location. A typical human dose for administration through routes such as intramuscular, is in the region of 200 ul to 750 ml, such as 400 to 600 ul, in particular about 500 ul, such as 500 ul.

If two liquids are intended to be combined, for example for co-formulation if the coronavirus spike antigen is in liquid form and the squalene emulsion adjuvant is in liquid form, the volume of each liquid may be the same or different. Volumes for combination will typically be in the range of 10:1 to 1:10, such as 2:1 to 1:2. Suitably the volume of each liquid will be substantially the same, such as the same. For example a 250 ul volume of coronavirus spike antigen in liquid form may be combined with a 250 ul volume squalene emulsion adjuvant in liquid form to provide a co-formulation dose with a 500 ul volume, each of the coronavirus spike antigen and squalene emulsion adjuvant being diluted 2-fold during the combination.

Squalene emulsion adjuvants may therefore be prepared as a concentrate with the expectation of dilution by a liquid coronavirus spike antigen containing composition prior to administration. For example, squalene emulsion adjuvant may be prepared at double-strength with the expectation of dilution by an equal volume of coronavirus spike antigen containing composition prior to administration.

The concentration of squalene at administration may be in the range 0.8 to 100 mg per ml, especially 1.2 to 48.4 mg per ml.

Coronavirus spike antigen and squalene emulsion adjuvant, whether intended for co-formulation or separate formulation, may be provided in the form of various physical containers such as vials or pre-filled syringes.

In some embodiments the coronavirus spike antigen, squalene emulsion adjuvant or kit comprising coronavirus spike antigen and squalene emulsion adjuvant is provided in the form of a single dose. In other embodiments the coronavirus spike antigen, squalene emulsion adjuvant or kit comprising coronavirus spike antigen and squalene emulsion adjuvant is provided in multidose form such containing 2, 5 or 10 doses. Multidose forms, such as those comprising 10 doses, may be provided in the form of a plurality of containers with single doses of one part (e.g. the coronavirus spike antigen) and a single container with multiple doses of the second part (e.g. squalene emulsion adjuvant) or may be provided in the form of a single container with multiple doses of one part (coronavirus spike antigen) and a single container with multiple doses of the second part (squalene emulsion adjuvant).

It is common where liquids are to be transferred between containers, such as from a vial to a syringe, to provide ‘an overage’ which ensures that the full volume required can be conveniently transferred. The level of overage required will depend on the circumstances but excessive overage should be avoided to reduce wastage and insufficient overage may cause practical difficulties. Overages may be of the order of 20 to 100 ul per dose, such as 30 ul or 50 ul. For example, a typical 10 dose container of doubly concentrated squalene emulsion adjuvant (250 ul per dose) may contain around 2.85 to 3.25 ml of squalene emulsion adjuvant.

Stabilisers may be present. Stabilisers may be of particular relevance where multidose containers are provided as doses of the final formulation(s) may be administered to subjects over a period of time.

Coronavirus spike antigen and squalene emulsion adjuvant in liquid form may be provided in the form of a multichamber syringe. The use of multi-chamber syringes provides a convenient method for the separate sequential administration of the coronavirus spike antigen and squalene emulsion adjuvant. Multi-chamber syringes may be configured to provide concurrent but separate delivery of the coronavirus spike antigen and squalene emulsion adjuvant, or they may be configured to provide sequential delivery (in either order).

In other configurations of multichambered syringes, the coronavirus spike antigen may be provided in dry form (e.g., freeze-dried) in one chamber and reconstituted by the squalene emulsion adjuvant contained in the other chamber before administration.

Examples of multi-chamber syringes may be found in disclosures such as WO2016/172396, although a range of other configurations are possible.

Formulations are preferably sterile.

Approaches for establishing strong and lasting immunity often include repeated immunisation, i.e. boosting an immune response by administration of one or more further doses. Such further administrations may be performed with the same immunogenic compositions (homologous boosting) or with different immunogenic compositions (heterologous boosting). The present invention may be applied as part of a homologous or heterologous prime/boost regimen, as either the priming or a/the boosting immunisation.

Administration of the coronavirus spike antigen and squalene emulsion adjuvant may therefore be part of a multi-dose administration regime. For example, the coronavirus spike antigen and squalene emulsion adjuvant may be provided as a priming dose in a multidose regime, especially a two- or three-dose regime, in particular a two-dose regime. The coronavirus spike antigen and squalene emulsion adjuvant may be provided as a boosting dose in a multidose regime, especially a two- or three-dose regime, such as a two-dose regime.

Priming and boosting doses may be homologous or heterologous. Consequently, the coronavirus spike antigen and squalene emulsion adjuvant may be provided as a priming dose and boosting dose(s) in a homologous multidose regime, especially a two- or three-dose regime, in particular a two-dose regime. Alternatively, the coronavirus spike antigen and squalene emulsion adjuvant may be provided as a priming dose or boosting dose in a heterologous multidose regime, especially a two- or three-dose regime, in particular a two-dose regime, and the boosting dose(s) may be different (e.g. a different coronavirus spike antigen; or an alternative antigen presentation such as protein or virally vectored antigen—with or without adjuvant, such as squalene emulsion adjuvant).

The time between doses may be two weeks to six months, such as three weeks to three months. Periodic longer-term booster doses may also be provided, such as every 2 to 10 years.

The squalene emulsion adjuvant may be administered to a subject separately from coronavirus spike antigen, or the adjuvant may be combined, either during manufacturing or extemporaneously, with coronavirus spike antigen to provide an immunogenic composition for combined administration.

Consequently, there is provided a method for the preparation of an immunogenic composition for use according to the present invention comprising a squalene emulsion adjuvant and coronavirus spike antigen, said method comprising the steps of:
(i) preparing a squalene emulsion adjuvant;
(ii) mixing the squalene emulsion adjuvant with a coronavirus spike antigen.

Also provided is a method for the preparation of an immunogenic composition for use according to the present invention comprising a squalene emulsion adjuvant and a coronavirus spike antigen, said method comprising the steps of:
(i) preparing a coronavirus spike antigen;
(ii) mixing the coronavirus spike antigen with squalene emulsion adjuvant.

To limit undesired degradation, squalene emulsions should generally be stored with limited exposure to oxygen e.g. in containers with limited headspace and/or by storage under nitrogen.

Throughout the specification, including the claims, where the context permits, the term “comprising” and variants thereof such as “comprises” are to be interpreted as including the stated element (e.g., integer) or elements (e.g., integers) without necessarily excluding any other elements (e.g., integers). Thus a composition “comprising” X may consist exclusively of X or may include something additional e.g. X+Y. In certain embodiments and for readability, the word “is” may be used as a substitute for “consists of” or “consisting of”.

The abbreviation, “e.g.” is derived from the Latin exempli gratia, and is used herein to indicate a non-limiting example. Thus, the abbreviation “e.g.” is synonymous with the term “for example.”

The word “substantially” does not exclude “completely” e.g. a composition which is “substantially free” from Y may be completely free from Y. Where necessary, the word “substantially” may be omitted from the definition of the invention.

The term “about” or “approximately” in relation to a numerical value x is optional and means, for example, x±10% of the given figure, such as x±5% of the given figure, in particular the given figure. Unless specifically stated otherwise, providing a numeric range (e.g., “25-30”) is inclusive of endpoints (i.e., includes the values 25 and 30). An endpoint of a range may be excluded by reciting “exclusive of lower endpoint” or “exclusive of upper endpoint”. Both endpoints may be excluded by reciting “exclusive of endpoints”.

As used herein, the singular forms “a,” “an” and “the” include plural references unless the content clearly dictates otherwise. The term “and/or” as used in a phrase such as “A and/or B” is intended to include “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone). Similarly, the word “or” is intended to include each of the listed elements individually as well as any combination of the elements (i.e., “or” herein encompasses “and”), unless the context clearly indicates otherwise.

As used herein, ng refers to nanograms, ug or μg refers to micrograms, mg refers to milligrams, mL or ml refers to milliliter, and mM refers to millimolar. Similar terms, such as um, are to be construed accordingly.

Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

An “immune response” is a response of a cell of the immune system (such as a B cell, T cell, or monocyte) to a stimulus (e.g., an antigen). An immune response can be a B cell response (or “humoral immune response”), which results in the production of specific antibodies, such as antigen-specific neutralizing antibodies. A “neutralizing antibody response” may be complement-dependent or complement-independent. A neutralizing antibody response may be cross-neutralizing (a neutralizing antibody generated against an antigen from one coronavirus, e.g., is neutralizing against the comparable antigen from another coronavirus). An immune response can also be a T cell response, such as a CD4+ T cell response or a CD8+ T cell response. In some cases, the response is specific for a particular antigen (that is, an “antigen-specific response”), in particular, a coronavirus spike antigen. If the antigen is derived from a pathogen, the antigen-specific response is a “pathogen-specific response” (e.g., a “MERS-CoV-specific immune response”, “a SARS-CoV-1-specific immune response”, or a “SARS-CoV-2-specific immune response”). A “protective immune response” is an immune response that reduces a detrimental function or activity of a pathogen, reduces infection by a pathogen (including cell entry), reduces cell-to-cell spread of a pathogen, and/or decreases symptoms (including death) that result from infection by the pathogen. A protective immune response can be measured, for example, by the inhibition of viral replication or plaque formation in a plaque reduction assay or ELISA-neutralization assay, or by measuring resistance to pathogen challenge in vivo. It may be further specified that the humoral immune response, CD4 T cell response, or CD8 T cell response is “at natural immunity”, “comparable to natural immunity”, or “above natural immunity”. It would be understood that what constitutes “natural immunity” is determined by analysis of patient subpopulations' immune responses to natural infection and whether or not a candidate vaccine elicits an immune response that is comparable to or greater than (above) natural immunity is a common consideration by regulatory bodies. Methods for measuring an immune response are known and may include, for measure of the humoral response, the Geometric Mean Titre (GMT) with 95% Confidence Interval (CI) of neutralizing antibodies and/or, for measure of the cell-mediated/cellular response, the concentration of T cell cytokines. For example, induction of proliferation or effector function of the particular lymphocyte type of interest (e.g., B cells, T cells, T cell lines, and T cell clones) may be assessed; for example, spleen cells from immunized mice can be isolated and the capacity of cytotoxic T lymphocytes to lyse autologous target cells that contain a polynucleotide that encodes the coronavirus spike antigen. In addition, T helper cell differentiation can be analyzed by measuring proliferation or production of TH1 (IL-2, TNF-α, or IFN-γ) cytokines and/or TH2 (IL-4 or IL-5) cytokines by ELISA or directly in CD4+ T cells by cytoplasmic cytokine staining and flow cytometry. Contemporary techniques for such analysis often include Enzyme-Linked Immunospot (ELIspot) and Flow Cytometry (FCM)-based detection. Certain cytokines are associated with certain classes of T cell(s) and, thus, the measure of those cytokines is associated with a cellular (T cell) immune response. Exemplary cytokines and their associated class of T cell(s) are below. Literature on detecting and quantifying an immune response includes: Plebanski, 2010; Todryk, 2018; Folds, 2003; Falchetti, 1998.

| Cytokines | Class of T cell |
|---|---|
| IFNgamma, TNFalpha, IL-2 | Th1 |
| IL-4, IL-5, IL-6, IL-9, IL-10, IL-13 | Th2 |
| IL-17 A/F, IL-22, IL-21, IL-25, IL-26 | Th17 |

“Immunogenicity” refers to an antigen or composition ability to induce an immune response. See generally, e.g., Ma, 2011. An “immunogenic composition” is a composition that, administered to a subject, will induce an immune response. As used herein, an immunogenic composition (e.g., a prophylactic or therapeutic vaccine composition) means that which is suitable for pharmaceutical use, including use for administration to a human subject.

An “effective amount” means an amount sufficient to cause the referenced outcome. An “effective amount” can be determined empirically and using known techniques in relation to the stated purpose. An “immunologically effective amount”, with respect to an antigen or immunogenic composition, is a quantity sufficient to elicit a measurable immune response in a subject (e.g., 1 to 100 μg of antigen). With respect to an adjuvant, an “adjuvanting effective amount” is a quantity sufficient to modulate an immune response. To obtain a protective immune response against a pathogen, it can require multiple administrations. So in the context of, for example, a protective immune response, an “immunologically effective amount” encompasses a fractional dose that contributes in combination with previous or subsequent administrations to attaining a protective immune response.

By “linked” it is meant the two or more referenced molecules or structures are connected, attached, fused, bound, or ligated. The two or more molecules and/or structures may be linked naturally (e.g., by the action of an endogenous enzyme and including the covalent or non-covalent bonds that naturally form between two proteins) or recombinantly (e.g., contacting two polynucleotides with a heterologous enzyme to ligate the polynucleotides together or recombinantly inserting one or more linkers between two proteins so that the proteins form a complex); and/or linked reversibly or irreversibly. For clarity, the two or more molecules and/or structures may be linked chemically (e.g., chemical conjugation of a protein and a sugar) or biologically (e.g., enzymatic conjugation of a protein and a sugar). “Linked” does not mean the two or more molecules and/or structures have to be next to each other (“adjacent”) without any other molecule or structure between them (“immediately adjacent to”).

“Operably linked” means two or more molecules are linked or attached (e.g., directly or indirectly in a covalent or non-covalent, perhaps reversible, manner) such that the function of the two or more molecules is maintained. In the context of a fusion/chimeric protein comprising, for example, a carrier (such as a nanoparticle, antibody, or antibody fragment) operably linked to a protein antigen, it would be understood that a variety of linkage techniques may be used and that “operably linked” would refer to the function of the nanoparticle (or antibody or antibody fragment) as carrier and of the protein as antigen being maintained.

“Purified” means removed from its natural environment and substantially free of impurities from that natural environment (such as other proteins. For clarity and as used herein, an antigen is a purified antigen (whether or not the word “purified” is recited). It is understood in the field that for an antigen to be suitable for pharmaceutical use (i.e., “pharmaceutically acceptable”), it must be appropriately purified (i.e., not crude). It would be further understood that “purified” is a relative term and that absolute (100%) purity is not required for, e.g., pharmaceutical use. A molecule may be at a purity of at least 85%, 86%, 87%, 88%, 89%, 90%, 91%, 92%, 93%, 94% or 95% of a composition's total proteinaceous mass (determined by, e.g., gel electrophoresis). Embodiments wherein coronavirus spike antigen is presented in the form of a nanoparticle may also comprise nanoparticle structural proteins. Methods of purification are known and include, e.g., various types of chromatography such as High Performance Liquid Chromatography (HPLC), hydrophobic interaction, ion exchange, affinity, chelating, and size exclusion; electrophoresis; density gradient centrifugation; or solvent extraction.

“Isolated” means removed from its natural environment and not linked to a recombinant molecule or structure (e.g., not bound to a recombinant antibody or antibody fragment) including not linked to a laboratory tool (e.g., not linked to a chromatography tool such as not bound to an affinity chromatography column). Hence, an “isolated coronavirus spike antigen” is not on the surface of a coronavirus-infected cell or within an infectious coronavirus virion or bound to a recombinant antibody or recombinant antibody fragment (which occurs in an ELISA assay, for example). It would be understood that an antigen being bound to an antibody or antibody fragment (through epitope recognition, for example) is different than an antigen being operably linked to an antibody or antibody fragment.

“Recombinant”, when used to describe a biological molecule or biological structure, means the biological molecule or biological structure is artificially produced (e.g., by laboratory methods), synthetic, and/or has a different structure and/or function than the molecule or structure from which it was obtained or than its wild type counterpart. For clarity, a recombinant molecule or recombinant structure that is synthetic may nonetheless function comparably to its wild type counterpart. A “recombinant protein/polypeptide” thereby encompasses a protein/polypeptide produced by expression of a recombinant polynucleotide. For clarification, a “purified protein” (e.g., a protein suitable for pharmaceutical use) is encompassed within the term “recombinant protein” because a purified protein is both artificially produced and has a different function than the crude protein (or extract or culture) from which it was obtained. A biological molecule or biological structure of the present invention may be described as “artificially produced”. “Heterologous” denotes that the two referenced biological molecules or biological structures are not naturally associated with each other (would not contact each other but-for the hand of man) or that the referenced biological molecule/structure is not in its natural environment. For example, when a polypeptide is in contact with or in a complex with another protein that it is not associated with in nature, the polypeptide may be referred to as “heterologous” (i.e., the polypeptide is heterologous to the protein).

“Reducing” means to lower or eliminate (i.e., “reduce/-ing” includes zero or 100% reduction). “Lowering” as used herein does not include zero (i.e., excludes 100% reduction or elimination). “Prevention” means to inhibit or stop (i.e., “prevent/-ing/-ion” includes zero or 100% blockage). “Inhibition” as used herein does not include zero (i.e., “inhibit/-ing/-ion” excludes 100% blockage or stopping).

Consistent with the official naming conventions in the art, the Severe Acute Respiratory Syndrome (SARS) betacoronavirus human pathogen which caused the international 2019/2020 pandemic may be referred to as “SARS-CoV-2” (Gorbalenya, 2020; see Wang, 2020, with previous names being “WH-Human1” (see Wu, 2020) and “2019-nCoV” (see Wrapp, 2020). The respiratory disease(s) caused by SARS-CoV2 may be referred to as “COVID-19” (Gorbalenya, 2020), e.g. viral pneumonia having exemplary symptoms of fever, cough, and/or dyspnea). For clarity, “SARS-CoV-1” is used herein to refer to the SARS betacoronavirus, lineage B human pathogen which caused an epidemic in 2002/2003 (see Li, 2005). What is “SARS-CoV-1” herein is usually referred to as just “SARS-CoV” in the art. “SARS-βCoV” may be used herein to refer to SARS betacoronaviruses in general (including MERS-CoV, SARS-CoV-1, and SARS-CoV-2). “SARS-β, BCoV” may be used to refer to SARS beta, lineage B coronaviruses in general (including SARS-CoV-1 and SARS-CoV-2).

Unless specifically stated, a process comprising a step of mixing two or more components does not require any specific order of mixing. Thus components can be mixed in any order. Where there are three components then two components can be combined with each other, and then the combination may be combined with the third component, etc.

### EXAMPLES

### Example 1—Squalene Emulsion Manufacture

Oil phase composed of squalene and D/L-alpha tocopherol was formulated under a nitrogen atmosphere. Aqueous phase, composed of modified phosphate buffered saline and polysorbate 80, was prepared separately. Oil and aqueous phases were combined at a ratio of 1:9 (volume of oil phase to volume of aqueous phase) before homogenisation and microfluidisation (three passes through a microfluidiser at around 15000 psi). The resulting emulsion was sterile filtered through two trains of two 0.5/0.2 um filters in series (i.e. 0.5/0.2/0.5/0.2).

A final content of ca 42.76 mg/ml squalene, 47.44 mg tocopherol and 19.44 mg/ml polysorbate 80 was targeted, i.e. double strength AS03_{A} based on a 500 ul dose volume.

Particle size and polydispersity was determined by DLS to be within the range 140 to 180 nm and less than 0.2 respectively. Squalene and tocopherol content was confirmed by HPLC and polysorbate 80 content by spectrophotometry to be within specification.

### REFERENCES

Arunachalam, P et al. Adjuvanting a subunit SARS-CoV-2 nanoparticle vaccine to induce protective immunity in non-human primates Version 1. bioRxiv. Preprint. 2021 Feb 11. doi: 10.1101/2021.02.10.430696.
Bakhshandeh, B et al. Mutations in SARS-CoV-2; Consequences in structure, function, and pathogenicity of the virus Microbial Pathogenesis (2021) doi: https://doi.org/10.1016/j.micpath.2021.104831.
Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9)
Bosch et al., The Coronavirus Spike Protein Is a Class I Virus Fusion Protein: Structural and Functional Characterization of the Fusion Core Complex J. Virol. 77:8801-8811 (2003).
Cathcart, A. et al. The dual function monoclonal antibodies VIR-7831 and VIR-7832 demonstrate potent in vitro and in vivo activity against SARS-CoV-2 bioRxiv preprint doi: https://doi.org/10.1101/2021.03.09.434607
Carmona Martinez A, et al. A phase 1, open-label safety and immunogenicity study of an AS03-adjuvanted trivalent inactivated influenza vaccine in children aged 6 to 35 months. Hum Vaccin Immunother. 2014 10(7):1959-1968.
Carter C, et al. A structure-function approach to optimizing TLR4 ligands for human vaccines, Clinical & Translation Immunology, 2016, 5, e108.
Gorbalenya, A et al., The species Severe acute respiratory syndrome-related coronavirus: classifying 2019-nCoV and naming it SARS-CoV-2, 2020 Nat. Microbiol. 5(4):536:544.
Cohet C, et al. Safety of AS03-adjuvanted Influenza Vaccines: A Review of the Evidence Vaccine 2019 37(23):3006-302.
Cui, J et al. Origin and evolution of pathogenic coronaviruses Nat. Rev. Microbiol. 2019 17(3):181-192.
Daugelaite et al. An Overview of Multiple Sequence Alignments and Cloud Computing in Bioinformatics, International Scholarly Research Notices, vol. 2013, Article ID 615630, 14 pages, 2013.
Dayhoff M, O et al. A model of evolutionary change in proteins, Atlas of Protein Sequence and Structure, 1978 vol 5, supp. 3.
Falchetti et al Determination of cytokine co-expression in individual splenic CD⁴⁺ and CD⁸⁺ T cells from influenza virus-immune mice, 1998, Immunology 95:346-351.
Follis et al. Furin cleavage of the SARS coronavirus spike glycoprotein enhances cell-cell fusion but does not affect virion entry, 2006 Virology 350:358-369.
Fox C. Squalene Emulsions for Parenteral Vaccine and Drug Delivery Molecules 2009 14:3286-3312.
Francica, J et al. Vaccination with SARS-CoV-2 Spike Protein and AS03 Adjuvant Induces Rapid Anamnestic Antibodies in the Lung and Protects Against Virus Challenge in Nonhuman Primates Version 1. bioRxiv. Preprint. 2021 Mar 2 doi: 10.1101/2021.03.02.433390.
Galson, J et al. Investigating the effect of AS03 adjuvant on the plasma cell repertoire following pH1 N1 influenza vaccination Scientific Reports Volume 6, Article number: 37229 (2016).
Garçon N, et al. Development and Evaluation of AS03, an Adjuvant System Containing α-Tocopherol and Squalene in an Oil-In-Water Emulsion Expert Rev Vaccines 2012 11:349-366.
Graham B., Rapid COVID-19 vaccine development Science 2020 368 (6494), 945-946.
Greaney, A. et al. Comprehensive mapping of mutations in the SARS-CoV-2 receptor-binding domain that affect recognition by polyclonal human plasma antibodies Cell Host & Microbe 2021 29:463-476 (https://doi.org/10.1016/j.chom.2021.02.003)
Hamid, M. M. A et al. Vaccination with Plasmodium knowlesi AMA1 formulated in the novel adjuvant co-vaccine HT™ protects against blood-stage challenge in rhesus macaques. PLoS One. 2011; 6(5):e20547.
Hilgers, L. A. et al. Sucrose fatty acid sulphate esters as novel vaccine adjuvant, Vaccine, 2006, 24 Suppl 2:S2-81-2.
Jackson L, et al. Effect of Varying Doses of a Monovalent H7N9 Influenza Vaccine With and Without AS03 and MF59 Adjuvants on Immune Response: A Randomized Clinical Trial JAMA 2015 314(3):237-246.
Kirchdoerfer et al., Stabilized coronavirus spikes are resistant to conformational changes induced by receptor recognition or proteolysis Sci. Rep. 8:15701 (2018).
Klucker M-F, et al. AF03, an Alternative Squalene Emulsion-Based Vaccine Adjuvant Prepared by a Phase Inversion Temperature Method J Pharm Sci. 2012 101(12):4490-4500.
Leroux-Roels, I et al. Priming with AS03A-adjuvanted H5N1 influenza vaccine improves the kinetics, magnitude and durability of the immune response after a heterologous booster vaccination: An open non-randomised extension of a double-blind randomised primary study Vaccine 2010 28:849-857
Letko, M. et al. Functional assessment of cell entry and receptor usage for SARS-CoV-2 and other lineage B betacoronaviruses Nat. Microbio. 2020 5:562-569.
Lodaya R, et al. Formulation Design, Optimization and In Vivo Evaluations of an α-Tocopherol-Containing Self-Emulsified Adjuvant System using Inactivated Influenza Vaccine J Control Release 2019 316:12-21.
Li, Structure, Function, and Evolution of Coronavirus Spike Proteins Annu. Rev. Virol. 3:237-261 (2016).
Li et al. Structure of SARS coronavirus spike receptor-binding domain complexed with receptor, 2005 Science 309:1864-1868.
Liu et al., Improvement of Pharmacokinetic Profile of TRAIL via Trimer-Tag Enhances Its Antitumor Activity in Vivo Sci Rep. 7:8953 (2017).
Ma et al., Envelope Deglycosylation Enhances Antigenicity of HIV-1 gp41 Epitopes for Both Broad Neutralizing Antibodies and Their Unmutated Ancestor Antibodies, 2011 PLoS Path. 7(9), e1002200.
Mahase, E. Covid-19: Where are we on vaccines and variants? BMJ 2021 372:n597.
Millet, J. K. and Whittaker, G, R., Host cell entry of Middle East respiratory syndrome coronavirus after two-step, furin-mediated activation of the spike protein, 2014 PNAS 111(42):15214-15219.
Morel S, et al. Adjuvant System AS03 Containing α-Tocopherol Modulates Innate Immune Response and Leads to Improved Adaptive Immunity Vaccine 2011 29:2461-2473.
Mullan, L. Pairwise sequence alignment—it's all about us!, 2005 Briefings in Bioinformatics 7(1):113-115.
Needleman and Wunsch, A general method applicable to the search for similarities in the amino acid sequence of two proteins, 1970 J. Mol. Biol. 48(3): 443-453.
O'Hagan D. MF59 Is a Safe and Potent Vaccine Adjuvant That Enhances Protection Against Influenza Virus Infection Expert Rev Vaccines 2007 6(5):699-710.
O'Hagan D, et al. The History of MF59(®) Adjuvant: A Phoenix That Arose From the Ashes Expert Rev Vaccines 2013 12(1):13-30.
Pallesen et al., Immunogenicity and structures of a rationally designed prefusion MERS-CoV spike antigen Proc. Natl. Acad. Sci. U.S.A. 114:E7348—E7357 (2017).
Pinto, D. et al. Cross-neutralisation of SARS-CoV-2 by a human monoclonal SARS-CoV antibody Nature 2020 583 290-295
Plebanski et al. The challenge of assessing infant vaccine responses in resource-poor settings, 2010 Expert Rev. Vaccines 9(6):596-600.
Podda A. The Adjuvanted Influenza Vaccines With Novel Adjuvants: Experience With the MF59-adjuvanted Vaccine Vaccine 2001 19:2673-2680.
Podda A and Giudice D. MF59-adjuvanted vaccines: increased immunogenicity with an optimal safety profile Expert Rev Vaccines 2003 2:197-203.
Public Health England, SARS-CoV-2 variants of concern and variants under investigation in England, Technical briefing 7.
Robert A. Meyers (ed.), Molecular Biology and Biotechnology: a Comprehensive Desk Reference, published by VCH Publishers, Inc., 1995 (ISBN 1-56081-569-8).
Richmond P et al., Safety and immunogenicity of S-Trimer (SCB-2019), a protein subunit vaccine candidate for COVID-19 in healthy adults: a phase 1, randomised, double-blind, placebo-controlled trial Lancet 2021; 397: 682-94.
Rice et al. EMBOSS: The European Molecular Biology Open Software Suite, 2000, Trends Genetics 16: 276-277; see also the World Wide Web at ebi.ac.uk/Tools/psa/emboss_needle.
Shah R, et al. The impact of size on particulate vaccine adjuvants. Nanomedicine (Lond) 2014 9:2671-2681.
Shah R, et al. The development of self-emulsifying oil-in-water emulsion adjuvant and an evaluation of the impact of droplet size on performance. J Pharm Sci. 2015 104:1352-1361.
Shah R, et al. The droplet size of emulsion adjuvants has significant impact on their potency, due to differences in immune cell-recruitment and -activation Scientific Reports 2019 9:11520
Sun, J et al. Analytical characterization of an oil-in-water adjuvant emulsion, AAPS PharmSciTech, 2017 18, 5, 1595-1604.
Tai et al. Characterization of the receptor-binding domain (RBD) of 2019 novel coronavirus: implication for development of RBD protein as a viral attachment inhibitor and vaccine, 2020 Cell. & Mol. Imm. 17, 613-620.
Tirado S M, Yoon K J. Antibody-dependent enhancement of virus infection and disease. Viral Immunol. 2003; 16(1):69-86.
Todryk, T Cell Memory to Vaccination, 2018 Vaccines (Basel) 6(4): 84.
Folds and Schmitz Clinical and laboratory assessment of immunity 2003 J. Allergy Clinical Immunology 111(2) Supplement 2: S702-S711.
van der Most, R et al. Seeking Help: B Cells Adapting to Flu Variability Science Translational Medicine 2014 Vol 6 Issue 246 246ps8
Walls A C et al. Unexpected Receptor Functional Mimicry Elucidates Activation of Coronavirus Fusion. Cell. 2019 Feb. 21; 176(5):1026-1039.e15. Walls, A. et al. Elicitation of Potent Neutralizing Antibody Responses by Designed Protein Nanoparticle Vaccines for SARS-CoV-2 Cell 183(5):1367-1382.
Wan et al. Molecular Mechanism for Antibody-Dependent Enhancement of Coronavirus Entry, 2020, J Virol 94:e02015-19.
Wang et al. Structural and Functional Basis of SARS-CoV-2 Entry by Using Human ACE2, 2020 Cell 181:894-904.
Wang et al. MERS-CoV spike protein: Targets for vaccines and therapeutics 2016, Antiviral Research 133: 165-177.
Wang, P. et al. Antibody Resistance of SARS-CoV-2 Variants B.1.351 and B.1.1.7 Nature https://doi.org/10.1038/s41586-021-03398-2 (2021).
Wang, Q et al. Structural and Functional Basis of SARS-CoV-2 Entry by Using Human ACE2 Cell 2020 181(4):894-904.
Wrapp et al., Cryo-EM structure of the 2019-nCoV spike in the prefusion conformation Science 367:1260-1263 (2020).
Wu et al. A new coronavirus associated with human respiratory disease in China, 2020 Nature 579:265-269.
Younis, S et al. Down selecting adjuvanted vaccine formulations: a comparative method for harmonized evaluation, BMC Immunol, 19, 6 (2018).
Younis, S et al. Identification of adjuvants for clinical trials performed with Plasmodium falciparum AMA1 in rabbits. BMC Immunol, 20, 25 (2019).
Yuan et al. Cryo-EM structures of MERS-CoV and SARS-CoV spike glycoproteins reveal the dynamic receptor binding domains, 2017 Nat. Comm. 8(15092).
Zheng B J et al. Studies of SARS virus vaccines, 2008 Hong Kong Med J 14(Suppl 4):S39-43. Du L. et al. 2009 Nat. Rev. Microbio. 7:226-236; Wang et al. 2016 Antiviral Research 133: 165-177. Handbook of Pharmaceutical Excipients (eds. Rowe, Sheskey, & Quinn) 6th edition, 2009 ISBN 978-0-85369-792-3
Light Scattering from Polymer Solutions and Nanoparticle Dispersions Schartl W. 2007 ISBN 978-E3-540-71950-2
Vaccine Design: The Subunit and Adjuvant Approach (chapter 10; Ott G, et al.) 1995 ISBN 978-1-4613-5737-7.
Vaccine Adjuvants: Preparation Methods and Research Protocols (chapter 12; Ott G, et al.) 2000 ISBN 0-89603-735-5.
New Generation Vaccines (eds. Levine et al.). 3rd edition, 2004. ISBN 0-8247-4071-8 (https://covid19.who.int/; accessed 17 Mar. 2021).
Humenza™ Summary of Product Characteristics
Fluad™ Summary of Product Characteristics
WO90/14837
WO2018/176103
WO2006/100109
WO2008/043774
WO2011/154444
WO2011/154443
WO2016/172396
WO2015/140138
WO2016/135154
WO2018/081318
WO2020/160080
EP 0 868 918 B1
US2007/0014805

1. A method for the prophylaxis of infection by a first coronavirus in a human subject, the method comprising administering to the subject (i) a coronavirus spike antigen derived from a second coronavirus, and (ii) a squalene emulsion adjuvant.

2. A method for inducing a cross-reactive immune response against a first coronavirus in a human subject, the method comprising administering to the subject (i) a coronavirus spike antigen derived from a second coronavirus, and (ii) a squalene emulsion adjuvant.

3-17. (canceled)

18. The method according to claim 1, wherein the squalene emulsion adjuvant has an average droplet size of less than 1 um.

19. (canceled)

20. The method according to claim 1, wherein the squalene emulsion adjuvant has a polydispersity of 0.5 or less.

21. The method according to claim 1, wherein the squalene emulsion adjuvant comprises a squalene emulsion adjuvant surfactant selected from poloxamer 401, poloxamer 188, polysorbate 80, sorbitan trioleate, sorbitan monooleate and polyoxyethylene 12 cetyl/stearyl ether either alone, in combination with each other or in combination with other surfactants.

22. The method according to claim 21, wherein the squalene emulsion adjuvant surfactant is selected from polysorbate 80, sorbitan trioleate, sorbitan monooleate and polyoxyethylene 12 cetyl/stearyl ether either alone, or in combination with each other.

23. The method according to claim 22, wherein the squalene emulsion adjuvant surfactant includes polysorbate 80.

24. (canceled)

25. The method according to claim 1, wherein the squalene emulsion adjuvant comprises two squalene emulsion adjuvant surfactants.

26. (canceled)

27. The method according to claim 1, wherein the amount of squalene in a single dose of the squalene emulsion adjuvant is 50 mg or less.

28-33. (canceled)

34. The method according to claim 1, wherein the weight ratio of squalene to surfactant in the squalene emulsion adjuvant is 0.73 to 6.6.

35-37. (canceled)

38. The method according to claim 1, wherein the squalene emulsion adjuvant does not comprise tocopherol.

39. The method according to claim 38, wherein the squalene emulsion adjuvant consists essentially of squalene, surfactant and water.

40. The method according to claim 38, wherein the squalene emulsion adjuvant comprises squalene, polysorbate 80, sorbitan trioleate and water.

41. (canceled)

42. The method according to either claim 40, wherein squalene emulsion adjuvant comprises citrate ions e.g. 10 mM sodium citrate buffer.

43-44. (canceled)

45. The method according to claim 40, wherein a single dose of the squalene emulsion adjuvant comprises 0.9 to 11 mg of squalene.

46-50. (canceled)

51. The method according to claim 38, wherein the squalene emulsion adjuvant comprises squalene, sorbitan monooleate, polyoxyethylene cetostearyl ether and water.

52-84. (canceled)

85. The method according to claim 1, wherein the squalene emulsion adjuvant comprises tocopherol.

86-87. (canceled)

88. The method according to claim 85, wherein the squalene emulsion adjuvant consists essentially of squalene, tocopherol, surfactant and water.

89-128. (canceled)

129. The method according to claim 1, wherein the subject is a naïve subject which has not previously been infected with or vaccinated against (e.g. not vaccinated against) a second coronavirus.

130. The method according to claim 1, wherein the subject is a primed subject which has previously been infected with or vaccinated against (e.g. vaccinated against) a coronavirus (e.g. a SARS-CoV-2).

131. (canceled)

132. The method according to claim 1, wherein the first and second coronaviruses are immunologically distinguishable with the level of spike protein specific antibodies in convalescent sera from a subject infected by one coronavirus being 2-fold or greater different from the level of spike specific antibodies for the other coronavirus.

133-241. (canceled)

242. The method according to claim 1, wherein the squalene emulsion adjuvant and the coronavirus spike antigen are administered within 12 hours of each other.

243-251. (canceled)
